(19) United States
(12) Patent Application Publication
(10) Pub. No.: US 2025/0368684 A1
(43) Pub. Date: Dec. 4, 2025

(54) NEW EGFRVIII-BINDING PEPTIDES, CONJUGATES AND USES THEREOF

(71) Applicant: NANOTHERA BIOSCIENCES, INC., Mount Laurel, NJ (US)

(72) Inventors: Sourour MANSOUR, Andresy (FR); Coralie LEBLEU, Breuillet (FR); Gauthier ERRASTI, Orsay (FR); Thomas DELACROIX, Mennecy (FR); Raj CHAKRABARTI, Moorestown, NJ (US); Indranil ADHYA, Durham (GB); Rama Krishna DUMPATI, Telangana (IN)

(73) Assignee: NANOTHERA BIOSCIENCES, INC., Mount Laurel, NJ (US)

(21) Appl. No.: 18/723,109

(22) PCT Filed: Dec. 22, 2022
(86) PCT No.: PCT/EP2022/087500

§ 371 Date: Jun. 21, 2024

Related U.S. Application Data
(60) Provisional application No. 63/293,464, filed on Dec. 23, 2021.

(30) Foreign Application Priority Data
Apr. 26, 2022 (EP) 22305624.3

(51) Int. Cl. C07K 7/08; A61K 38/00

(52) U.S. Cl. CPC C07K 7/08; A61K 38/00

(57) ABSTRACT

The present invention relates to a peptide of 12 to 30 amino acids, said peptide comprising the sequence SEQ ID NO: 1 represented by:

(SEQ ID NO: 1)
V-X1-X2-R-X3-E-W-X4-X5-X6-Y-W,

wherein each of X1, X2, X3, X4, X5, and X6 independently corresponds to any amino acid, and
wherein said peptide binds to the EGF receptor variant III (EGFRvIII), as well as products incorporating such peptide and uses thereof.

### CROSS REFERENCE TO RELATED APPLICATIONS

This application is a National Stage of International Application No. PCT/EP2022/087500 filed Dec. 22, 2022, claiming priority based on U.S. Provisional Application No. 63/293,464 filed Dec. 23, 2021 and claiming priority based on European patent Application No. 22305624.3 filed Apr. 26, 2022.

### INCORPORATION BY REFERENCE OF SEQUENCE LISTING

The instant application contains a Sequence Listing which has been filed electronically in xml format and is hereby incorporated by reference in its entirety. Said xml copy, created on Jun. 20, 2024, is named Sequence_listing.xml and is 58.4 KB in size.

### FIELD OF THE INVENTION

The present invention relates to the field of molecular biology. In particular, the invention relates to a peptide, and a consensus sequence thereof, capable of binding, with a high specificity, the Epithelial Growth Factor Receptor variant III (EGFRvIII), a cancer-specific cell surface marker. The peptide according to the invention can be used as a tumor targeting peptide in a variety of applications such as delivering cytotoxic agents and/or labeling molecules and therefore finds utility in cancer treatment and/or diagnosis.

### BACKGROUND

Cancer diseases are characterized by the uncontrolled proliferation of abnormal cells. The development of a cancer cell, a process called carcinogenesis, is multifactorial, i.e. linked to a combination of several factors, whose role and importance in the development of the disease are variable. Carcinogenesis results from an accumulation of several irreversible alterations and takes place in three phases: initiation, promotion and proliferation. Furthermore, a primary tumor can eventually spread to other locations through a process known as metastasis.

Treatments for cancer include surgery, radiation therapy, interventional radiology, immunotherapy as well as chemotherapy, including targeted cancer therapies. Targeted cancer therapies aim to deliver drugs or other substances that block the growth and spread of cancer by interfering with cancer specific molecules, thereby reducing the risks of unwanted side effects. In this context, there is a need to develop new agents capable of specifically targeting tumors cells but not normal wild type cells.

One of the few known cancer-specific cell surface markers is the epidermal growth factor (EGF) tyrosine kinase receptor mutation variant III (EGFRvIII) (Pedersen et al., 2001). EGFRvIII does not contain a ligand-binding domain and is constitutively active (Guo et al., 2015; Huang et al., 1997). Although the kinase activity of EGFRvIII is much weaker than that for ligand-activated full-length EGFR, this weak constitutive kinase activity has been reported as enough to confer growth advantage to tumors (Batra et al., 1995; Moscatello et al., 1996). EGFRvIII is present in a number of human malignancies: ovarian cancer, breast cancer (Boohaker et al., 2012) and glioblastoma (Ge et al., 2002). However, in contrast to EGFR WT, which is expressed in most mouse and human healthy cells, EGFRvIII has never been identified in normal tissues. Thus, EGFRvIII acts as a potential target to achieve efficient drug uptake via receptor-mediated endocytosis (Dhankhar et al., 2010).

Tumor-targeting ligands such as peptides and antibodies may effectively aid to deliver certain cytotoxic agents (either biological or synthetic) to the tumor cells, thereby improving therapeutic efficacy while limiting the exposure of normal tissues to the cytotoxic agents (Lin N U, 2014).

Therapeutic approaches have included the use of unarmed MAbs (Lorimer, 2002; Modjtahedi et al., 2003), radiolabeled MAbs (Omidfar et al., 2004), MAbs conjugated to immunotoxins (Kuan et al., 2001; Archer et al., 1999) or boronated dendrimers (Yang et al., 2008). Small peptides (3-5 kDa) that selectively recognize tumor cells have advantages over antibodies (150 kDa), as due to their much lower size, they are easy to synthesize and modify (Cloughesy et al., 2014), have higher cell membrane penetration, and possess less immunogenicity.

Two short peptides intended for tumor cells targeting have been described in the prior art (Denholt et al., 2009; Denholt et al., 2011; Mao et al., 2017): the ‘FALGEA’ (SEQ ID NO:5) peptide is described as binding to both EGFR WT and EGFRvIII, while the ‘YHWYGYTPENVI’ (SEQ ID NO: 6), discovered from the GE11 peptide) has only been described as binding to EGFR WT, while its potential ability to bind EGFRvIII is unknown. Thus, there is a need to develop new peptides capable of binding to EGFRvIII with high specificity, compared to EGFR WT, and that allow to deliver certain cytotoxic agents into the tumor and tumor cells for cancer targeted therapy, or deliver certain labels into the tumor and tumor cells for diagnosis purposes.

Phage display of peptides is widely used for the development of peptide ligands. An important step in the phage selection of peptides is the comparison of sequences and the identification of consensus motifs. Consensus sequences can provide valuable information about the binding site of peptides. Peptides sharing the same consensus motif likely bind to the same surface region of the target protein and form similar molecular interactions. If isolated ligands are to be used as leads in drug development, multiple consensus sequences are desired as parallel development of several peptides leads to increases in the success rate of the development program. Peptides of one consensus sequence might share unfavorable properties such as poor solubility or low proteolytic stability, therefore drug development based on a single peptide may lead to a dead end. Hence, there is a need to develop new consensus sequences to allow the parallel development of drugs based on multiple possible EGFRvIII-binding peptides.

In this context, the Inventors have identified novel peptides, capable to bind EGFRvIII with a good specificity over EGFR WT. Starting from one of these peptides, the Inventors were able to identify positions in the peptide that are more or less important for specific binding to EGFRvIII and thus to develop a new consensus sequence thereby providing an array of new EGFRvIII-binding peptides. Interestingly, these peptides can be used as targeting peptides, with application in assisting specific delivery of cytotoxic drugs, either biological or synthetic, specifically into the tumor vasculature, tumor microenvironment and/or directly into cancer cells, thereby improving therapeutic efficacy while limiting the exposure of normal tissues to the cytotoxic agents. The peptides may also be used to deliver labels specifically to cancer cells for diagnosis purposes.

### SUMMARY OF THE INVENTION

The present invention relates to a peptide of 12 to 30 amino acids, said peptide comprising the sequence SEQ ID NO: 1 represented by:

(SEQ ID NO: 1)
V-X1-X2-R-X3-E-W-X4-X5-X6-Y-W,

wherein each of X1, X2, X3, X4, X5, and X6 independently corresponds to any amino acid, and
wherein said peptide binds to the EGF receptor variant III (EGFRvIII).

In a preferred embodiment,
a) each of X1 and X2 is a hydrophobic amino acid, preferably selected from the group consisting of: A, V, I, L, M, F, Y, W, G, and P; and/or
b) each of X3, X4, X5, and X6 is a hydrophilic amino acid, preferably selected from the group consisting of: R, H, K, D, E, S, T, N, Q, C, and U.

Preferably, the peptide according to the invention has higher affinity for EGFRvIII than for wild type EGF receptor (EGFR WT).

In another preferred embodiment, X4 is selected from the group consisting of: T, S, N, Q, C, and U, preferably said peptide comprises the sequence SEQ ID NO: 2 represented by:

(SEQ ID NO: 2)
V-X1-X2-R-X3-E-W-S-X5-X6-Y-W,

In another preferred embodiment, X5 is selected from the group consisting of: T, S, N, Q, C, and U, preferably said peptide comprises the sequence SEQ ID NO: 3 represented by:

(SEQ ID NO: 3)
V-X1-X2-R-X3-E-W-S-T-X6-Y-W,.

In another preferred embodiment,
a) X1 is selected from the group consisting of: V, A, I, M, G, P and L, preferably X1 is L,
b) X2 is selected from the group consisting of: V, A, I, M, G, P and L, preferably X2 is G,
c) X3 is selected from the group consisting of: E, D, Q, and N, preferably X3 is E,
d) X6 is selected from the group consisting of: T, S, N, Q, C, and U, preferably X6 is S, or
e) any combination of a) to d) above.

In another preferred embodiment, said peptide comprises the sequence SEQ ID NO: 4 represented by:

(SEQ ID NO: 4)
V-L-G-R-E-E-W-S-T-S-Y-W.

In another preferred embodiment, said peptide consists of 12 to 25 amino acids, preferably 12 to 20 amino acids, more preferably 12 to 15 amino acids, more preferably 12 to 13 amino acids, and most preferably said peptide consists of 12 amino acids.

The present invention also relates to a nucleic acid encoding the peptide according to the invention.

The present invention also relates to a vector comprising a nucleic acid according to the invention.

The present invention also relates to a host cell comprising the nucleic acid or the vector according to the invention.

The present invention also relates to a conjugate comprising the peptide according the invention linked to another entity, preferably said peptide and the other entity are covalently linked to each other, optionally via a linker.

In a preferred embodiment, said linker is:
a) a non-cleavable linker preferably selected from the group consisting of: aliphatic chains, PEG, and poly(amino acid) derivatives; or
b) a cleavable linker preferably selected from the group consisting of: chemical labile linkers, disulfide-containing reducible linkers, enzymatically cleavable linkers, and chemically and/or thermally cleavable.

In a preferred embodiment, said other entity is selected from a radiolabel, a fluorophore, another peptide, a polymer, a copolymer, a nanoparticle, a drug, or any combination thereof.

In a preferred embodiment,
a) said radiolabel is selected from the group consisting of: ¹⁸F, ⁶⁴Cu, and ⁹⁹mTc;
b) said fluorophore is selected from the group consisting of: fluorescein isothiocyanate (FITC), phycoerythrin (PE), Allophycocyanin (APC), fluorophores from the cyanine family, fluorophores from AlexaFluor family, fluorophores from Atto family, and fluorophores from Dy family;
c) said other peptide is 3-8 amino acids long, such as GGGGG, CGGGG, CGGGS, and Beta-alanine, or is a short peptide tags such as His-tag, Myc-tag or GST-tag; d) said polymer is selected from the group consisting of: polyethylene glycol (PEG), poly(amino acid)s and polysaccharides;
e) said copolymer is selected from the group consisting of: copolymers of PEG and another polymer, notably an hydrophobic polymer;
f) said nanoparticle is selected form the group consisting of:
   polymeric nanoparticles, including polymeric micelles, polymersomes, and polyplexes; and
   lipid-based nanoparticles with conjugation on various lipids, for example liposomes; and
   inorganic nanoparticles based for example on: selenium, silica, gold; or
g) said drug is an anti-tumoral drug.

The present invention also relates to the use of the peptide according to the invention or the conjugate according to the invention as an EGFRvIII-binding molecule, preferably for:
a) detecting or quantifying EGFRvIII-expressing cells or EGFRvIII-expressing secreted extracellular vesicles (SEV) in a biological sample in vitro or ex vivo;
b) detecting or quantifying EGFRvIII-expressing cells or EGFRvIII-expressing SEVs in a subject in vivo;
b) sorting EGFRvIII-expressing cells or EGFRvIII-expressing SEVs from a biological sample in vitro;
c) in vitro targeting of EGFRvIII; and
d) in vitro delivery of an entity to an EGFRvIII-expressing cell, wherein said other entity is preferably selected from a radiolabel, a fluorophore, another peptide, a polymer, a copolymer, a nanoparticle, a drug, or any combination thereof.

The present invention also relates the peptide according to the invention or the conjugate according to the invention, or a pharmaceutical composition comprising said peptide or conjugate and a pharmaceutically acceptable vehicle, for use in the treatment of cancer, preferably said cancer is an EGFRvIII-expressing cancer, more preferably selected from ovarian cancer, breast cancer and glioblastoma.

### FIGURES LEGEND

FIG. 1. Procedure for the analysis of sequencing data applying python scripts. First, reads are separated into several files according to their barcode. Second, low-quality sequences are removed from the dataset, and remaining sequences are translated and sorted by abundance.

FIG. 2. Fluorescence intensity quantification measured by flow cytometry. 293T, EGFR WT and EGFRvIII cell lines were incubated either with anti-M13, anti-EGFR WT or anti-EGFRvIII as primary antibodies. Cell counts in % versus PE signal: when incubated with primary+secondary antibody (solid line), isotype labelling+secondary antibody (dashed line) or secondary antibody only (dotted (grey) line).

FIG. 3. Fluorescence intensity at 525 nm versus FITC-Ahx-peptide molar concentration. Data shown for FITC-Ahx-FALGEA (circle, dark grey), FITC-Ahx-YHWYGYTPENVI (diamond, light grey), and FITC-Ahx-VLGREEWSTSYW (square, black). Slopes of the linear regressions were used to determine correction factors to normalize MFI obtained by flow cytometry.

FIG. 4. The total number of reads and sequences in each round of biopanning. The Y-axis represents the number of reads (left) or sequences (right). The X-axis represents each round of biopanning.

FIG. 5. The top 5 peptides enriching over the rounds of biopanning. The Y-axis represents the enrichment value and the X-axis represents the peptide sequences.

FIG. 6. VLGREEWSTSYW-M13 binding analysis by flow cytometry. 293T, EGFR WT and EGFRvIII cell lines were incubated with the VLGREEWSTSYW-M13 phage displayed peptide. Cell counts in % versus PE signal: when incubated with primary+secondary antibody (solid line), isotype labelling+secondary antibody (dashed line) or secondary antibody only (dotted (grey) line).

FIG. 7. Quantitative cellular binding of FITC-Ahx-peptides on 293T, WT and vIII cells by flow cytometry. A,B,C) 293T cells (dotted (grey) line), EGFR WT cells (dashed line) and EGFRvIII cells (solid line) were incubated with 10 UM of FITC-Ahx-peptides: A) FALGEA, B) YHWYGYTPENVI, C) VLGREEWSTSYW for 20 min at RT. A,B,C) Cell counts in % versus FITC signal. D) Normalized mean fluorescence intensities (MFI) of each peptide for each cell line. E) Ratio for each peptide of the normalized MFI obtained for vIII cell line over WT cell line.

### DETAILED DESCRIPTION

### General Definitions

The terms “comprising”, “including” or “containing” must be understood as inclusive and the recited feature is open-ended, it does not exclude additional, unrecited elements notably further amino acids. Hence, in the context of the present invention, the terms “comprising”, “including” or “containing” should be understood as meaning that at least all of the recited elements (notably the recited amino acids) must but present but other elements (such as other amino acids) that are not mentioned may also be present.

The term “consisting of” must be understood as closed-ended and excluding any further element (notably amino acids) which is not specifically recited in the feature.

### EGFRvIII Binding Peptides

As illustrated in Example 1, the Inventors have identified novel peptides, capable to bind EGFRvIII with a good specificity over EGFR WT. In addition, as illustrated in Example 2, starting from one of these peptides (V-L-G-R-E-E-W-S-T-S-Y-W (SEQ ID NO: 4)), the Inventors were able to identify positions in the peptide that are more or less important for specific binding to EGFRvIII and thus to develop a new consensus sequence thereby providing an array of new EGFRvIII-binding peptides. The positions determined as critical for binding to EGFRvIII are positions 1, 4, 6-7, and 11-12 of V-L-G-R-E-E-W-S-T-S-Y-W (SEQ ID NO: 4).

In a first aspect, the present invention thus relates to a peptide of 12 to 30 amino acids, said peptide comprising the sequence SEQ ID NO: 1 represented by:

(SEQ ID NO: 1)
V-X1-X2-R-X3-E-W-X4-X5-X6-Y-W,

wherein each of X1, X2, X3, X4, X5, and X6 independently corresponds to any amino acid, and
wherein said peptide binds to the EGF receptor variant III (EGFRvIII).

By “peptide” it is meant polymers of amino acid residues linked by peptide bonds. A peptide may be linear, branched or cyclic. A peptide may comprise natural amino acids and/or non-natural amino acids and may be interrupted by non-amino acid residues. As a general indication and not related thereto in the present application, if the amino acid polymer contains more than 50 amino acid residues, it is preferably referred to as a polypeptide or a protein, whereas if the polymer consists of 50 or fewer amino acids, it is preferably referred to as a peptide.

The peptide according to the invention comprises a “minimal sequence”, defined as the sequence necessarily included into the claimed peptide. The minimal sequence of wider scope defined herein is consensus sequence SEQ ID NO: 1, but other more limited consensus sequences (SEQ ID NO:2 or 3) or a fixed sequence (SEQ ID NO:4) are defined below as preferred minimal sequences.

In addition, the peptide according to the invention consists of 12 to 30 amino acids, meaning that at most 18 additional amino acids may be present in N-terminal and/or in C-terminal of SEQ ID NO: 1 or any other minimal sequence (any one of SEQ ID NO:2 to 4). Indeed, the presence of a few additional amino acids in N-terminal and/or in C-terminal of the minimal sequence does not hamper binding to EGFRvIII. However, the number of additional amino acids in N-terminal and/or in C-terminal of the minimal sequence is preferably even lower than 18, and the peptide according to the invention therefore preferably consists of only 12 to 25 amino acids, 12 to 20 amino acids, more preferably 12 to 18 amino acids (at most 6 additional amino acids may be present in N-terminal and/or in C-terminal of the minimal sequence), 12 to 16 amino acids (at most 4 additional amino acids may be present in N-terminal and/or in C-terminal of the minimal sequence), 12 to 15 amino acids (at most 3 additional amino acids may be present in N-terminal and/or in C-terminal of the minimal sequence), 12 to 14 amino acids (at most 2 additional amino acids may be present in N-terminal and/or in C-terminal of the minimal sequence), 12 to 13 amino acids (at most 1 additional amino acids may be present in N-terminal and/or in C-terminal of the minimal sequence). Most preferably, the peptide according to the invention consists of the minimal sequence (i.e., consists of any one of SEQ ID NO: 1 to 4).

By “natural amino acid” it is meant an amino acid that may be incorporated into a protein upon messenger RNA translation. Natural amino acids are also referred to as proteinogenic amino acids. These are L-alanine, L-arginine, L-asparagine, L-aspartate, L-cysteine, L-glutamate, L-glutamine, glycine, L-histidine, L-isoleucine, L-leucine, L-lysine, L-methionine, L-phenylalanine, L-proline, L-pyrrolysine, L-selenocysteine, L-serine, L-threonine, L-tryptophan, L-tyrosine, and L-valine.

By “non-natural amino” acid it is meant an amino acid that may not be incorporated into a protein upon messenger RNA translation. As a non-limiting example, these non-natural amino acids can be D-amino acids, phosphorylated amino acids, carboxylated amino acids, acetylated amino acids, metabolic intermediates such as: ornithine, citrulline, argininosuccinate, homoserine, homocysteine, and others.

Consensus sequence SEQ ID NO:1 contains 6 fixed natural amino acids. The peptide according to the invention thus comprises at least 6 natural amino acids, but may preferably comprise at least 7 natural amino acids, such as at least 8, 9, 10, 11, or 12 natural amino acids. In a preferred embodiment, the peptide according to the invention comprises natural amino acids only.

By “EGFRvIII” or “EGFR variant III” it is meant a mutant of the epidermal growth factor receptor. EGFRvIII mutated receptor lacks amino acids residues 6-273 in the extracellular domain, and deletion of those 268 amino acids creates a junction site with a new glycine residue between amino acids 5 and 274, compared with the “wild-type EGF receptor” or “EGFR WT”. EGFRvIII is present in number of human malignancies such as ovarian cancer, breast cancer and glioblastoma. In particular, EGFRvIII is not expressed in non-malignant wild type cells.

At each variable position in consensus sequence SEQ ID NO: 1, the peptide according to the invention may comprise a hydrophobic amino acid or a hydrophilic amino acid.

By “hydrophobic amino acid” it is meant an amino acid which possesses a side-chain that tends to be repelled by an aqueous environment (i.e. water). These side chains are composed mostly of carbon and hydrogen, and have very small dipole moments. In the context of the present invention, it is considered that hydrophobic natural amino acids are glycine (Gly, G), alanine (Ala, A), valine (Val, V), leucine (Leu, L), isoleucine (Ile, I), proline (Pro, P), phenylalanine (Phe, F), methionine (Met, M), tyrosine (Tyr, Y) and tryptophan (Trp, W). Under physiological conditions, most protein normally lie within an aqueous medium.

For this reason, hydrophobic amino acids are generally found buried within the hydrophobic core of a protein, or, in the case of membranous-proteins, within the lipid portion of the membrane. According to the present invention, hydrophobic amino acids are preferably selected from the group consisting of: A, V, I, L, M, F, Y, W, G, and P.

By “hydrophilic amino acid” it is meant an amino acid which possesses a side-chain that preferably resides in an aqueous environment (i.e. attracts in water). These amino acids have a polar nature. In the context of the present invention, it is considered that hydrophilic natural amino acids are arginine (Arg, R), histidine (His, H), lysine (Lys, K), aspartic acid (Asp, D), glutamic acid (Glu, E), serine (Ser, S), threonine (Thr, T), asparagine (Asn, N), glutamine (Gln, Q), cysteine (Cys, C), and Selenocysteine (Sec, U). According to the present invention are preferably selected from the group consisting of: R, H, K, D, E, S, T, N, Q, C, and U.

However, hydrophobic amino acids are preferred at some variable positions of the minimal sequence, while hydrophilic amino acids are preferred at other variable positions of the minimal sequence. In particular:
a) X1 is preferably a hydrophobic amino acid, more preferably selected from the group consisting of: A, V, I, L, M, F, Y, W, G, and P;
b) X2 is preferably a hydrophobic amino acid, more preferably selected from the group consisting of: A, V, I, L, M, F, Y, W, G, and P;
c) X3 is preferably a hydrophilic amino acid, more preferably selected from the group consisting of: R, H, K, D, E, S, T, N, Q, C, and U;
d) X4 is preferably a hydrophilic amino acid, more preferably selected from the group consisting of: R, H, K, D, E, S, T, N, Q, C, and U;
e) X5 is preferably a hydrophilic amino acid, more preferably selected from the group consisting of: R, H, K, D, E, S, T, N, Q, C, and U;
f) X6 is preferably a hydrophilic amino acid, more preferably selected from the group consisting of: R, H, K, D, E, S, T, N, Q, C, and U; or
g) Any combination of a) to f) above, in particular the combination of all of a) to f) above.

In a preferred embodiment, the present invention relates to a peptide of 12 to 30 amino acids, said peptide comprising or consisting of the sequence SEQ ID NO: 1 represented by:

(SEQ ID NO: 1)
V-X1-X2-R-X3-E-W-X4-X5-X6-Y-W,,

wherein, each of X1 and X2 is a hydrophobic amino acid, preferably selected from the group consisting of: A, V, I, L, M, F, Y, W, G, and P; and/or (preferably and)
wherein each of X3, X4, X5, and X6 is a hydrophilic amino acid, preferably selected from the group consisting of: R, H, K, D, E, S, T, N, Q, C, and U.

According to the present invention, the peptide of sequence SEQ ID NO 1 binds to the EGF receptor variant III (EGFRvIII). A few peptides are known from the art, as binding at least to EGFR WT. In particular, the ‘FALGEA’ (SEQ ID NO:5) peptide is able to target both EGFR WT and EGFRvIII, while the ‘YHWYGYTPENVI’ (SEQ ID NO:6) peptide is only described as binding EGFR WT. In this context, the inventors have surprisingly found that the peptide according to the invention binds to EGFRvIII cells not only with a high efficiency but also with a good specificity compared to EGFR WT cells. Furthermore, said peptide displays low unspecific interactions.

This higher affinity for the EGFRvIII is particularly advantageous since it allows the use of the peptide according to the invention for the specific targeting of cancer cells expressing EGFRvIII (as normal cells do not express this variant, contrary to EGFR WT that may be expressed in normal cells), notably for the development of new tools for the treatment and/or the diagnosis of cancer.

Hence, in a favorite embodiment, the peptide according to the invention has higher affinity for EGFRvIII than for wild type EGF receptor (EGFR WT).

While positions 2-3, 5 and 8-10 of V-L-G-R-E-E-W-S-T-S-Y-W (SEQ ID NO: 4) have been found not to be critical for binding to EGFRvIII, position 8 was found to be slightly involved in binding to EGFRvIII, and the presence of particular amino acid residues at this position is thus preferred for higher binding to EGFRvIII.

In a preferred embodiment, the present invention thus relates to a peptide of 12 to 30 amino acids (or any other range or value of length mentioned above), said peptide comprising or consisting of the sequence SEQ ID NO: 1; wherein, X4 is selected from the group consisting of: T, S, N, Q, C, and U.

Still, preferably X4 is a serine(S).

Hence, in a preferred embodiment, the peptide according to the invention comprises or consists of the sequence SEQ ID NO: 2 represented by:

(SEQ ID NO: 2)
V-X1-X2-R-X3-E-W-S-X5-X6-Y-W,.

In more limited consensus sequence SEQ ID NO:2, X1, X2, X3, X5 and X6 may be selected from any amino acid (natural or non-natural). However, as in more general consensus sequence SEQ ID NO: 1:
a) X1 is preferably a hydrophobic amino acid, more preferably selected from the group consisting of: A, V, I, L, M, F, Y, W, G, and P;
b) X2 is preferably a hydrophobic amino acid, more preferably selected from the group consisting of: A, V, I, L, M, F, Y, W, G, and P;
c) X3 is preferably a hydrophilic amino acid, more preferably selected from the group consisting of: R, H, K, D, E, S, T, N, Q, C, and U;
d) X5 is preferably a hydrophilic amino acid, more preferably selected from the group consisting of: R, H, K, D, E, S, T, N, Q, C, and U;
e) X6 is preferably a hydrophilic amino acid, more preferably selected from the group consisting of: R, H, K, D, E, S, T, N, Q, C, and U; or
f) Any combination of a) to e) above.

Even more preferably:
each of X1 and X2 is a hydrophobic amino acid, preferably selected from the group consisting of: A, V, I, L, M, F, Y, W, G, and P; and/or (preferably and).
each of X3, X5, and X6 is a hydrophilic amino acid, preferably selected from the group consisting of: R, H, K, D, E, S, T, N, Q, C, and U.

While even less important for binding to EGFRvIII than position 8, position 9 of V-L-G-R-E-E-W-S-T-S-Y-W (SEQ ID NO: 4) was found to be slightly involved in binding to EGFRvIII, and the presence of particular amino acid residues at this position is thus preferred for higher binding to EGFRvIII.

In a preferred embodiment, the present invention thus relates to a peptide of 12 to 30 amino acids (or any other range or value of length mentioned above), said peptide comprising or consisting of the sequence SEQ ID NO: 1 or SEQ ID NO: 2; wherein, X5 is selected from the group consisting of: T, S, N, Q, C, and U.

Still, preferably X5 is a threonine (T).

Hence, in a preferred embodiment, the peptide according to the invention comprises or consists of the sequence SEQ ID NO: 3 represented by:

(SEQ ID NO: 3)
V-X1-X2-R-X3-E-W-S-T-X6-Y-W,.

In more limited consensus sequence SEQ ID NO: 3, X1, X2, X3, and X6 may be selected from any amino acid (natural or non-natural). However, as in more general consensus sequence SEQ ID NO: 1:
a) X1 is preferably a hydrophobic amino acid, more preferably selected from the group consisting of: A, V, I, L, M, F, Y, W, G, and P;
b) X2 is preferably a hydrophobic amino acid, more preferably selected from the group consisting of: A, V, I, L, M, F, Y, W, G, and P;
c) X3 is preferably a hydrophilic amino acid, more preferably selected from the group consisting of: R, H, K, D, E, S, T, N, Q, C, and U;
d) X6 is preferably a hydrophilic amino acid, more preferably selected from the group consisting of: R, H, K, D, E, S, T, N, Q, C, and U; or
e) Any combination of a) to d) above.

Even more preferably:
each of X1 and X2 is a hydrophobic amino acid, preferably selected from the group consisting of: A, V, I, L, M, F, Y, W, G, and P; and/or (preferably and)
each of X3 and X6 is a hydrophilic amino acid, preferably selected from the group consisting of: R, H, K, D, E, S, T, N, Q, C, and U.

While positions 2-3, 5 and 10 of V-L-G-R-E-E-W-S-T-S-Y-W (SEQ ID NO: 4) have been found to be the less important for binding to EGFRvIII, the presence of particular amino acids at these positions may still further ensure high binding to EGFRvIII.

In a preferred embodiment, the present invention thus relates to a peptide of 12 to 30 amino acids (or any other range or value of length mentioned above), said peptide comprising or consisting of the sequence SEQ ID NO: 1, SEQ ID NO: 2 or SEQ ID NO: 3; wherein:
a) X1 is selected from the group consisting of: V, A, I, M, G, P and L, preferably X1 is L,
b) X2 is selected from the group consisting of: V, A, I, M, G, P and L, preferably X2 is G,
c) X3 is selected from the group consisting of: E, D, Q, and N, preferably X3 is E,
d) X6 is selected from the group consisting of: T, S, N, Q, C, and U, preferably X6 is S, or
e) any combination of a) to d) above.

Based on the preferred amino acids present in positions 2 (X1), 3 (X2), 5 (X3), 8 (X4), 9 (X5), and 10 (X6), the present invention preferably relates to a peptide of 12 to 30 amino acids (or any other range or value of length mentioned above), said peptide comprising or consisting of the sequence SEQ ID NO: 1 represented by:

(SEQ ID NO: 1)
V-X1-X2-R-X3-E-W-X4-X5-X6-Y-W,

wherein:
   a) X1 is selected from the group consisting of: V, A, I, M, G, P and L, preferably X1 is L, and/or
   b) X2 is selected from the group consisting of: V, A, I, M, G, P and L, preferably X2 is G, and/or
   c) X3 is selected from the group consisting of: E, D, Q, and N, preferably X3 is E, and/or
   d) X4 is selected from the group consisting of: T, S, N, Q, C, and U, preferably X4 is S, and/or
   e) X5 is selected from the group consisting of: T, S, N, Q, C, and U, preferably X5 is T, and/or
   f) X6 is selected from the group consisting of: T, S, N, Q, C, and U, preferably X6 is S,
and,
wherein said peptide binds to the EGF receptor variant III (EGFRvIII).

In particular, the present invention also relates to a peptide of 12 to 30 amino acids (or any other range or value of length mentioned above), said peptide comprising or consisting of the sequence SEQ ID NO: 1 represented by:

(SEQ ID NO: 1)
V-X1-X2-R-X3-E-W-X4-X5-X6-Y-W,

wherein:
   a) X1 is selected from the group consisting of: V, A, I, M, G, P and L,
   b) X2 is selected from the group consisting of: V, A, I, M, G, P and L,
   c) X3 is selected from the group consisting of: E, D, Q, and N,
   d) X4 is selected from the group consisting of: T, S, N, Q, C, and U,
   e) X5 is selected from the group consisting of: T, S, N, Q, C, and U, and
   f) X6 is selected from the group consisting of: T, S, N, Q, C, and U,
and wherein said peptide binds to the EGF receptor variant III (EGFRvIII).

The present invention also relates to a peptide of 12 to 30 amino acids (or any other range or value of length mentioned above), said peptide comprising or consisting of the sequence SEQ ID NO: 2 represented by:

(SEQ ID NO: 2)
V-X1-X2-R-X3-E-W-S-X5-X6-Y-W,

wherein:
   a) X1 is selected from the group consisting of: V, A, I, M, G, P and L, preferably X1 is L, and/or
   b) X2 is selected from the group consisting of: V, A, I, M, G, P and L, preferably X2 is G, and/or
   c) X3 is selected from the group consisting of: E, D, Q, and N, preferably X3 is E, and/or
   d) X5 is selected from the group consisting of: T, S, N, Q, C, and U, preferably X5 is T, and/or
   e) X6 is selected from the group consisting of: T, S, N, Q, C, and U, preferably X6 is S,
and wherein said peptide binds to the EGF receptor variant III (EGFRvIII).

In particular, the present invention also relates to a peptide of 12 to 30 amino acids (or any other range or value of length mentioned above), said peptide comprising or consisting of the sequence SEQ ID NO: 2 represented by:

(SEQ ID NO: 2)
V-X1-X2-R-X3-E-W-S-X5-X6-Y-W,

wherein:
   a) X1 is selected from the group consisting of: V, A, I, M, G, P and L,
   b) X2 is selected from the group consisting of: V, A, I, M, G, P and L,
   c) X3 is selected from the group consisting of: E, D, Q, and N,
   d) X5 is selected from the group consisting of: T, S, N, Q, C, and U, and
   e) X6 is selected from the group consisting of: T, S, N, Q, C, and U,
and wherein said peptide binds to the EGF receptor variant III (EGFRvIII).

The present invention also relates to a peptide of 12 to 30 amino acids (or any other range or value of length mentioned above), said peptide comprising or consisting of the sequence SEQ ID NO: 3 represented by:

(SEQ ID NO: 3)
V-X1-X2-R-X3-E-W-S-T-X6-Y-W,

wherein:
   a) X1 is selected from the group consisting of: V, A, I, M, G, P and L, preferably X1 is L, and/or
   b) X2 is selected from the group consisting of: V, A, I, M, G, P and L, preferably X2 is G, and/or
   c) X3 is selected from the group consisting of: E, D, Q, and N, preferably X3 is E, and/or
   d) X6 is selected from the group consisting of: T, S, N, Q, C, and U, preferably X6 is S,
and wherein said peptide binds to the EGF receptor variant III (EGFRvIII).

In particular, the present invention also relates to a peptide of 12 to 30 amino acids (or any other range or value of length mentioned above), said peptide comprising or consisting of the sequence SEQ ID NO: 3 represented by:

(SEQ ID NO: 3)
V-X1-X2-R-X3-E-W-S-T-X6-Y-W,

wherein:
   a) X1 is selected from the group consisting of: V, A, I, M, G, P and L,
   b) X2 is selected from the group consisting of: V, A, I, M, G, P and L,
   c) X3 is selected from the group consisting of: E, D, Q, and N,
   d) X6 is selected from the group consisting of: T, S, N, Q, C, and U,
and wherein said peptide binds to the EGF receptor variant III (EGFRvIII).

In a favorite embodiment, the peptide according to invention comprises or consists of the sequence SEQ ID NO: 4 represented by:

(SEQ ID NO: 4)
V-L-G-R-E-E-W-S-T-S-Y-W.

### Nucleic Acids

The present invention also relates to a nucleic acid (herein also called nucleic or nucleotide sequence or polynucleotide) encoding at least one peptide as described above, notably a peptide comprising or consisting of the sequence SEQ ID NO: 1, SEQ ID NO: 2 SEQ ID NO: 3, or SEQ ID NO: 4.

By “Nucleic acid” it is meant a polymer of any length of deoxyribonucleic acid (DNA), or polydeoxyribonucleotides, including, but not limited to, complementary DNAs or cDNAs, genomic DNAs, plasmids, vectors, viral genomes, isolated DNA, probes, primers, and any mixtures thereof; or a polymer of any length of ribonucleic acid (RNA), or polyribonucleotides, including but not limited to messenger RNAs or mRNAs, antisense RNAs; or mixed polyribo-polydeoxyribonucleotides. They include single or double-stranded, linear or circular, natural or synthetic polynucleotides. In addition, a polynucleotide may include non-natural nucleotides and may be interrupted by non-nucleotide components.

Because of degeneration of the genetic code, all the different nucleic sequences encoding a particular amino acid sequence are within the scope of the invention. In particular, the sequence of a nucleic acid according to the invention may be optimized to promote the expression thereof in a host cell, in humans, or a transgenic non-human animal of interest. Indeed, there are in general several three-nucleotide combinations encoding the same amino acid (except for methionine and tryptophan), called synonymous codons. However, some of these combinations are in general used preferentially by a cell or a given organism (this is referred to as genetic code usage bias).

This preference depends notably on the producing organism or cell. Consequently, when a protein derived from one or more organisms is produced in a heterologous organism or a cell of such a heterologous organism, it may be useful to modify the nucleic sequence encoding the protein to use mainly the preferred codons of the heterologous organism. Data are available in the literature concerning the use of codons preferred by different species and a person skilled in the art knows how to optimize the expression of a given protein in a heterologous organism or a cell of a heterologous organism.

### Vectors

The present invention also relates to a vector comprising a nucleic acid according to the invention. Such a vector comprises the elements necessary for the expression of said nucleic sequence(s), and notably a promoter, a transcription initiation codon, termination sequences, and suitable transcription regulatory sequences. These elements vary according to the host used for the expression and are easily selected by persons skilled in the art based on their general knowledge.

The vector can notably be a plasmid vector, viral vector or a bacteriophage vector. It may be used to clone or express the nucleic acids according to the invention.

The one skilled in the art would routinely know and find vectors able to be used in the context of the invention, including the transcription unit to be used.

### Host Cells

The present invention also relates to a host cell comprising a nucleic acid according to the invention or a vector according to the invention.

The host cell may be of prokaryotic or eukaryotic origin, and may in particular be selected from bacterial, insect, plant, fungus, yeast or mammalian cells. The peptide according to the invention may then be produced by culturing the host cell under suitable conditions.

A host cell according to the invention can notably be obtained by transforming a cell line by the expression vector for the peptide according to the invention, and separating the various cell clones obtained. The transformed cell line is preferably of eukaryotic origin, and may in particular be selected from insects, plants, yeast, or mammalian cells. Suitable cell lines available for peptide production notably include cell lines selected from Chinese hamster ovary (CHO) cells, Baby hamster kidney (BHK) fibroblasts, murine lymphoid cell lines (NSO and Sp2/O), Human embryonic kidney (HEK293) cells and Human embryonic retinal (PER.C6) cells.

### Method for Producing the Peptide

Another aspect of the present invention relates to a method for producing a peptide according to the invention, and as described above, comprising at least the steps of:
a) culturing a host cell according to the invention under conditions suitable to produce said peptide; and
b) purifying said peptide from the cultured host cells.

Advantageously, the host cell is as described above.

The one skilled in the art would routinely know how to identify the culture conditions suitable for producing the peptide of step a). In particular, the skilled person will know which cell culture medium is most appropriate with regard to the type of cell used for producing the peptide. The skilled person will also know how to adjust the temperature, pH, amounts of O₂ and CO₂ and other parameters, depending on the cell type used for peptide production.

Peptide purification steps useful in step b) are also well known in the art, and notably include reversed-phase high performance liquid chromatography (RP-HPLC), flash chromatography, ion-exchange chromatography, hydrophobic interaction chromatography, gel filtration chromatography, size exclusion chromatography, and hydrophilic interaction chromatography.

### Conjugates

The peptide of the invention advantageously offers access to cancer cells and tumors, through specific binding to EGFRvIII. It is therefore very advantageous to use the peptide of the invention for the preparation of conjugates which can be used to deliver cargo molecules into the tumor.

Hence, another aspect of the present invention relates to a conjugate comprising the peptide according to the invention (as described above), linked to another entity.

The peptide of the invention and the other entity may be linked to each other covalently or non-covalently.

In particular, when the peptide of the invention is conjugated to metal or mineral nanoparticles, it may be non-covalently linked to such metal or mineral nanoparticles, as their metal or mineral composition allows for such non-covalent linkage through adsorption, electrostatic interactions, hydrophobic interactions/hydrogen bonding.

However, in most cases, the peptide of the invention and the other entity will preferably be covalently linked to each other.

### Linkers

According to particular embodiments, the peptide of the invention and the other entity are covalently linked to each other via a linker.

According to some embodiments, the linker is a non-cleavable linker. Preferably, said non-cleavable linker is selected from the group consisting of: aliphatic chains (e.g. 6-aminohexanoic acid (Ahx), SMCC), PEG derivatives (e.g. ADO, Mal-PEG-NHS), and poly(amino acid) derivatives (e.g. polyGly).

According to some other embodiments, the linker is a cleavable linker. Preferably, said cleavable linker is selected from the group consisting of: chemical labile linkers (e.g. MHH), disulfide-containing reducible linkers (e.g. DSDM, sulfo-SPDB), enzymatically cleavable linkers (e.g. MC-VC-PABC, GBC), and chemically and/or thermally cleavable (e.g. biotin and/or streptavidin).

### Entities Linked to the Peptides

The peptide of the invention may advantageously be conjugated to many types of other entities depending on the type of application contemplated. Said other entity may notably be selected from a radiolabel, a fluorophore, another peptide, a polymer, a copolymer, a nanoparticle, a drug, biotin and/or streptavidin or any combination thereof.

### Radiolabels

Peptides according to the invention conjugated to a radiolabel may be used either for diagnostic purposes (in order to detect EGFRvIII-expressing cancer cells) or for therapeutic purposes (for targeted radiotherapy).

In an embodiment, the peptide according to the invention may thus be linked (preferably covalently linked) to a radiolabel suitable for diagnosis, preferably selected from the group consisting of: ¹⁸F, ⁶⁴Cu, ⁹⁹mTc, ¹²³I, ⁶⁸Ga, and ¹¹¹In; and preferably selected from the group consisting of: ¹⁸F, ⁶⁴Cu, and ⁹⁹mTc.

In another embodiment, the peptide according to the invention may thus be linked (preferably covalently linked) to a radiolabel suitable for targeted radiotherapy, preferably selected from the group consisting of: ¹⁸F, ⁶⁴Cu, ⁹⁹mTc, ¹²³I, ⁶⁸Ga, and ¹¹¹In; and preferably selected from the group consisting of: ¹⁸F, ⁶⁴Cu, and ⁹⁹mTc.

More generally, the peptide according to the invention may thus be linked (preferably covalently linked, optionally via a linker) to a radiolabel selected from the group consisting of: ¹⁸F, ⁶⁴Cu, ⁹⁹mTc, ¹²³I, ⁶⁸Ga, and ¹¹¹In; and preferably selected from the group consisting of: ¹⁸F, ⁶⁴Cu, and ⁹⁹mTc.

The peptide according to the invention may be conjugated to such radiolabels using methods known in the art. For instance:
suitable methods for radiolabeling a peptide with ¹⁸F are disclosed in Denholt et al., 2011 or disclosed in Li et al., 2020;
suitable methods for radiolabeling a peptide with ⁶⁴Cu are disclosed in Dejesus et al., 2012 or disclosed in Striese et al., 2018;
suitable methods for radiolabeling a peptide with ⁹⁹mTc are disclosed in Rahmanian et al., 2017 or disclosed in Jiao et al., 2020.

### Fluorophores

Peptides according to the invention conjugated (preferably covalently linked, optionally via a linker) to a fluorophore may be used for diagnostic purposes (in order to detect EGFRvIII-expressing cancer cells).

The terms “fluorescent label”, “fluorescent group”, “fluorescent compound”, “fluorescent dye”, and “fluorophore”, as used herein, are synonymous and refer to compounds or moieties that absorb light energy at a defined excitation wavelength and emit light energy at a different wavelength.

Examples of fluorophores to which the peptide of the invention may be conjugated include, but are not limited to:
fluorophores of the Alexa Fluor family (including Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680),
fluorophores from the Atto family (including Atto 488, Atto 550, Atto 647),
fluorophores of the BODIPY family (including BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665),
fluorophores of the Cascade family (including Cascade Blue and Cascade Yellow),
Coumarin-based fluorophores (including Aminomethylcoumarin Acetate (AMCA), AMCA-S, Coumarin 6, Coumarin 343, Dialkylaminocoumarin, Hydroxycoumarin, and Methoxycoumarin),
Cyanine-based fluorophores (including Cy3, Cy5, Cy3.5, Cy5.5, Allophycocyanin (APC)),
fluorophores from Dy family (Dyomics, including DY-350, DY-488, DY-550, DY-700),
Fluorescein-based fluorophores (including 6-Carboxyfluorescein (6-FAM), Fluorescein, fluorescein isothiocyanate (FITC), 4′,5′-Dichloro-2′,7′-dimethoxy-fluorescein, Naphthofluorescein, 2′,4′,5′,7′-Tetra-bromosulfone-fluorescein, 6-Carboxy-4′,5′-Dichloro-2′,7′-Dimethoxyfluorescein (6-JOE)),
Fluorescein-derived fluorophores (including eosin, erythrosin
IRDyes (including IRD40, IRD 700, IRD 800),
fluorophores of the Oregon family (including Oregon Green 488, Oregon Green 500, and Oregon Green 514),
Phycoerythrin (PE),
Pyrene,
Rhodamine-based fluorophores (including Carboxyrhodamine 6G carboxy-X-rhodamine (ROX), Lissamine rhodamine B, Rhodamine B, Rhodamine 6G, Rhodamine Green, Rhodamine Red, Tetramethyl-rhodamine (TMR), Carboxytetramethylrhodamine (TAMRA)),
fluorophores of the Texas Red family (including Texas Red, Texas Red-X).

While any fluorophore of interest may be used, said fluorophore may notably be selected from the group consisting of: fluorescein isothiocyanate (FITC), phycoerythrin (PE), Allophycocyanin (APC), fluorophores from the cyanine family (cyanine 3 (Cy3), cyanine 5 (Cy5) and others), fluorophores from AlexaFluor family, fluorophores from Atto family, and fluorophores from Dy family (Dyomics).

Methods for conjugating a peptide to a fluorophore are known in the art (see e.g., Hossein-Nejad-Ariani et al., 2019; or Mao et al., 2017), and a skilled person will know how to choose the best method depending on the specific selected fluorophore.

### Other Peptides

Peptides according to the invention may also be conjugated (preferably covalently linked, optionally via a linker) to another peptide.

Said other peptide is preferably a short peptide, in particular the other peptide is preferably 3-8 amino acids long.

While any other peptide of interest may be conjugated to the peptide of the invention, a particular type of other peptide of interest for conjugation to the peptide of the invention is the group of flexible peptide linkers, such as GGGGG, CGGGG, CGGGS, and Beta-alanine (2 to 6 repeats). Other types of other peptide of interest include short peptide tags (such as a 6-histidine tag, a FLAG tag, a Myc tag, or a GST tag).

While other types of conjugation may be used, a peptide of the invention will preferably be conjugated or linked to another peptide by direct peptide fusion, the conjugate being a fusion comprising or consisting of, from N-terminal to C-terminal: peptide of the invention—other peptide or other peptide-peptide of the invention.

Such fusion peptide may be produced by direct chemical peptide synthesis. Methods for the chemical peptide synthesis are well known in the art an notably include liquid phase synthesis, solid-phase synthesis, and microwave-assisted peptide synthesis. These methods are reliable and advantageously allow the direct synthesis of large amount of a fusion of the peptide according to the invention with another peptide.

Such fusion may be recombinantly produced, for instance by transfecting a host cell with a nucleic acid or a vector comprising a nucleic acid encoding the fusion. The present invention thus also relates to a nucleic acid or a vector comprising a nucleic acid encoding a fusion of the peptide according to the invention with another peptide. The present invention also relates to a host cell comprising a nucleic acid or a vector encoding a fusion of the peptide according to the invention with another peptide.

### Polymers and Copolymers

Peptides according to the invention may also be conjugated (preferably covalently linked, optionally via a linker) to polymers or copolymers. Such conjugation to polymers or copolymers may be useful for increasing half-life in vivo or for further preparation of polymeric nanoparticles.

While any polymer of interest may be conjugated to the peptide of the invention, said polymer may preferably be selected from the group consisting of: polyethylene glycol (PEG), poly(amino acid) s, and polysaccharides (such as hyaluronic acid (HA)).

By “poly(amino acid)”, it is referred to any polymer consisting of two or more amino acids as monomeric units covalently linked by peptide bonds.

Similarly, while any copolymer of interest may be conjugated to the peptide of the invention, said copolymer may preferably be selected from copolymers of PEG and another polymer (as used herein, a copolymer of PEG and another polymer is intended to mean a copolymer comprising PEG and another polymer, which may further comprise other chemical groups, such as groups permitting conjugation to the peptide according to the invention), notably an hydrophobic polymer, and more preferably selected from the group consisting of: polyethylene glycol-polylactide (PEG-PLA), poly(ethylene glycol)-b-poly(D,L-lactide-co-glycolide) (PEG-PLGA), poly(ethylene glycol)-b-poly(ε-caprolactone) (PEG-PCL), poly(ethylene glycol)-b-poly(trimethylene carbonate-co-dithiolane trimethylene carbonate) (PEG-P (TMC-DTC)), and polyethyleneimine-poly(ethylene glycol) (PEI-PEG).

Methods for conjugating a peptide to a polymer or copolymer are known in the art and a skilled person will know how to select the best method depending on the selected type of polymer or copolymer.

For instance, in the case where the peptide according to the invention is conjugated or linked to a poly(amino acid), conjugation may be performed by recombinant fusion (as disclosed above for conjugation to another peptide). The conjugate is then a fusion comprising or consisting of, from N-terminal to C-terminal: peptide of the invention-poly(amino acid) or poly(amino acid)-peptide of the invention, and may be produced by recombinant technology, for instance by transfecting a host cell with a nucleic acid or a vector comprising a nucleic acid encoding the fusion. The present invention thus also relates to a nucleic acid or a vector comprising a nucleic acid encoding a fusion of the peptide according to the invention with a poly(amino acid). The present invention also relates to a host cell comprising a nucleic acid or a vector encoding a fusion of the peptide according to the invention with a poly(amino acid).

When the peptide according to the invention is conjugated or linked to PEG or a copolymer of PEG and another polymer, conjugation may be performed by coupling chemistries, click coupling, peptide synthesis from PEG chains, polymer synthesis from peptide, chemical conjugations (such as: amine reactive coupling, reductive amination, thiol reactive coupling, disulfide formation, copper-catalyzed alkyne-azide coupling (CuAAC), strain-promoted alkyne-azide coupling (SPAAC), inverse electron-demand Diels-Alder reaction (IEDDA), hydrazone formation, oxime formation, and polymerization), enzymatic conjugations (such as: transglutaminase-mediated, and peroxidase mediated, photo-conjugation (such as: photo-thiol-′ene′, and photo-acrylate crosslinking) these methods and others are notably described in Dehn, S., et al., 2011; lan W. Hamley, 2014; and Hersh, J. et al., 2021.

When the peptide according to the invention is conjugated or linked to polysaccharides (such as hyaluronic acid (HA)), conjugation may be performed by coupling chemistries as described in Mero et al., 2014.

### Nanoparticles

Peptides according to the invention may also be conjugated (preferably covalently linked, optionally via a linker) to a nanoparticle. Indeed, many types of nanoparticles are being developed for anticancer therapy or cancer diagnosis.

By “nanoparticle” it is meant an object characterized by a diameter between 1 and 400 nm, preferably between 1 and 100 nm.

While any type of nanoparticle of interest may be used, the nanoparticle conjugated or linked to the peptide of the invention may preferably be selected form the group consisting of:
polymeric nanoparticles;
lipid-based nanoparticles with conjugation on various lipids; and
inorganic nanoparticles; or

By “polymeric nanoparticle”, it is referred to a nanoparticle which 3-dimensional structure is made of a polymeric material. Polymeric nanoparticles notably include polymeric micelles, polymersomes, and polyplexes.

By “polymeric micelle” it is meant an object characterized by a core-shell structure with a hydrophobic core and hydrophilic shell, formed by assembly of amphiphilic copolymers.

By “polymersome” it is meant a polymer-based vesicle.

By “vesicle” it is meant an object with a structure of a bilayer enclosing an aqueous compartment, formed by assembly of amphiphilic molecules such as phospholipids (liposome) or amphiphilic copolymers (polymersome).

By “polyplex” it is meant a polymeric system containing a complexed nucleic acid (eg DNA or RNA), formed by electrostatic interactions between cationic groups of a polymer and negatively charged nucleic acid.

While any appropriate polymeric material may be used for preparing polymeric nanoparticles conjugated or linked to a peptide according to the invention, the polymeric material polymeric material may preferably be selected from:
copolymers of PEG and another polymer, such as poly(ethylene glycol)-b-poly(lactic acid) (PEG-PLA), poly(ethylene glycol)-b-poly(D,L-lactide-co-glycolide) (PEG-PLGA), poly(ethylene glycol)-b-poly(ε-caprolactone) (PEG-PCL), poly(ethylene glycol)-b-poly(trimethylene carbonate-co-dithiolane trimethylene carbonate) (PEG-P (TMC-DTC)), polyethyleneimine-poly(ethylene glycol) (PEI-PEG), and
polysaccharides, such as hyaluronic acid (HA).

The peptide according to the invention may be covalently conjugated or linked to a polymeric nanoparticle by methods well known from the skilled person in the art, notably those described above for conjugation to polymers and copolymers.

Furthermore, the nanoparticles can be conjugated to the peptide of the invention by way of pre-functionalization or post-functionalization. In the case of pre-functionalization, the peptide according to the invention is conjugated to a polymer having a reactive function (refered to as ‘F-polymer’) resulting in a peptide-polymer, i.e. a pre-functionalized polymer. In this situation, the nanoparticles are prepared with a mixture of polymer (not functionalized) and peptide-polymer (pre-functionalized). Alternatively, in the case of post-functionalization, the nanoparticles may be prepared with a mixture of polymer (non-functionalized) and ‘F-polymer’ resulting in ‘F-nanoparticles’ and then the peptide is conjugated to the F-nanoparticles.

By “lipid-based nanoparticle”, it is referred to a nanoparticle which 3-dimensional structure is made of lipids. Lipid-based nanoparticles notably include liposomes.

By “liposome” it is meant a lipid-based vesicle (as defined above).

The peptide according to the invention may be covalently conjugated or linked to a lipid-based nanoparticle by methods well known from the skilled person in the art.

In this context, pre-functionalization and post-functionalization (as described above) can be used. An alternative method is also described for the preparation of liposomes, said method involves a both pre and post-functionalization since the peptide-lipid is synthesized on one side and (non-functionalized) liposomes are formed separatly. In a subsequent step, liposomes and peptide-lipids are incubated together so that the latter are inserted into the liposome bilayer (Cheng, L. et al., (2014); Riaz, M. K. et al., (2018)).

For example, liposomes with 1,2-distearoyl-sn-glycero-3-phosphoethanolamine-N-[maleimide (polyethylene glycol)-2000] (DSPE-PEG2000-Mal) may be conjugated to a peptide according to the invention. This may be performed by methods well known from the skilled person in the art such as by maleimide-thiol coupling through cystein amino-acid (Cheng, L. et al., (2014); Xu, W.-W. et al., (2017)).

By “inorganic nanoparticle”, it is referred to a nanoparticle which 3-dimensional structure is made of an inorganic material, including metals (such as gold) and mineral materials (such as those including selenium or silica).

In the case of inorganic nanoparticles, the peptide according to the invention may be covalently or non-covalently conjugated or linked to the inorganic nanoparticle. Non-covalent linkage/conjugation may notably be performed by methods well known from the skilled person in the art. In particular, the peptide may be covalently bound to an inorganic nanoparticle stabilizer that is not covalently bound to said nanoparticle.

Covalent linkage/conjugation may notably be performed by methods well known from the skilled person in the art, such as ligand exchange, chemical conjugation, and chemical reduction (Li, X. et al., (2021)).

### Magnetic Particles

Peptides according to the invention may also be conjugated (preferably covalently linked, optionally via a linker) to a magnetic particle. Magnetic particles can be used in the context of cancer diagnosis notably via Magnetic Immunoassay (MIA). Methods for conjugating a peptide to a magnetic particle are known in the art (see e.g., Li, X. et al., (2021)).

By “magnetic particles” or “magnetic beads” it is meant an object made of magnetic nanometric-sized iron oxide particles encapsulated or glued together with polymers. These magnetic beads are characterized by a diameter between about 35 nm and about 4.5 μm. Due to their magnetic component, magnetic particles exhibit a unique quality referred to as superparamagnetism in the presence of an externally applied magnetic field.

### Drugs

Peptides according to the invention may also be conjugated (preferably covalently linked, optionally via la linker) to a drug.

While any drug of interest may be used, an anti-tumoral drug may preferably be used.

While any anti-tumoral drug of interest may be used, the anti-tumoral drug may preferably be selected from the group consisting of:
beta tubulin inhibitors (e.g. taxanes including paclitaxel),
tyrosine kinase inhibitors (e.g. brigatinib),
alkylating agent (e.g. ifosfamide),
antimetabolites (e.g. Decitabine),
anti-tumor antibiotics (e.g. anthracyclins including doxorubicin),
topoisomerase inhibitors (e.g. irinotecan),
mitotic inhibitors (e.g. taxanes including paclitaxel and docetaxel, vinca alkaloids including vinblastine),
corticosteriods (e.g. prednisone or dexamethasone),
hormone therapy,
immunotherapy,
immunomodulators,
other chemotherapy drug (e.g. all-trans retinoic acid, asparaginase, eribulin, hydroxyurea), and
RNA interference (e.g. siRNAs, amiRNAs).

### Nanoparticles

As nanoparticles are particularly developed as new anticancer treatments, the present invention also relates to a nanoparticle comprising one or more peptide(s) according to the invention displayed at its external surface. In a particular embodiment, the nanoparticles comprise at least one of the active compounds described above as a payload.

The nanoparticle may be selected from any type of nanoparticle disclosed above in the section directed to conjugates.

### Magnetic Particles

As magnetic particles are useful in the context of cancer diagnosis, the present invention also relates to a magnetic particle comprising one or more peptide(s) according to the invention displayed at its external surface.

### Compositions

Anticancer treatment is one of the uses intended for the peptide and conjugate according to the invention, and the present invention thus also relates to a pharmaceutical composition comprising any peptide or conjugate according to the invention (as disclosed above), and a pharmaceutically acceptable vehicle.

By a “pharmaceutically acceptable vehicle” it is meant a material that is not biologically or otherwise undesirable, i.e., the material may be administered to a subject without causing any undesirable biological effects or interacting in a deleterious manner with any of the other components of the pharmaceutical composition in which it is contained. The vehicle would naturally be selected to minimise any degradation of the active ingredient and to minimise any adverse side effects in the subject, as would be well known to one of skill in the art. The one skilled in the art would routinely know how to select some suitable pharmaceutical vehicle which include without limitation, e.g., water (including sterile and/or deionized water), suitable buffers (such as PBS), physiological saline, or the like.

### Uses and Methods

The peptide or conjugate according to the invention may be used in various contexts for cancer diagnosis or treatment, including in in vitro or ex vivo methods, therapeutic methods and diagnosis methods.

### In Vitro and Ex Vivo Methods

The peptide or conjugate according to the invention may firstly be used in various in vitro or ex vivo methods.

In particular, the present invention also relates to the use of any peptide or conjugate according to the invention (as disclosed above) as an EGFRvIII-binding molecule, preferably for:
a) detecting or quantifying EGFRvIII-expressing cells or EGFRvIII-expressing secreted extracellular vesicles (SEV) in a biological sample in vitro or ex vivo;
b) sorting EGFRvIII-expressing cells or EGFRvIII-expressing SEVs from a biological sample in vitro;
c) in vitro targeting of EGFRvIII; and
d) in vitro delivery of an entity to an EGFRvIII-expressing cell, wherein said other entity is preferably selected from a radiolabel, a fluorophore, another peptide, a polymer, a copolymer, a nanoparticle, a drug, or any combination thereof.

As used herein, a “biological sample” is any sample that can be collected from a subject. A “biological sample” may notably be a cancer sample or a sample suspected to be a cancer sample. A “cancer sample” refers to a sample that comprises cancer cells but may also comprise a proportion of healthy cells. Cancer samples notably include cancer biopsies (solid or liquid depending on the type of cancer), sample taken from a surgical cancer resection therapy, or any biological fluid (including but not limited to, whole blood, serum, plasma, sputum, nasopharyngeal swabs, urine, stool, cerebrospinal fluid, saliva, gastric secretions, semen, seminal fluid, tears, brain fluid) or tissue that may contain circulating cancer cells. A “sample suspected to be a cancer sample” is any sample disclosed above from a subject that is not yet confirmed as having a cancer or any sample from a cancer subject that comprises cells from a cell type different from the primary cancer and is suspected to contain metastasis cells.

By “subject” it is meant a mammal, human or animal, preferably human, male or female.

The present invention also relates to a method for in vitro delivery of an entity to an EGFRvIII-expressing cell, comprising contacting an EGFRvIII-expressing cell with any conjugate according to invention, as disclosed above.

The present invention also relates to a method for sorting EGFRvIII-expressing cells or EGFRvIII-expressing SEVs from a biological sample in vitro, comprising:
a) contacting a biological sample with any peptide or conjugate according to the invention; and
b) separating cells or SEVs bound to the peptide or conjugate from cells or SEVs not bound to the peptide or conjugate.

### Therapeutic Methods

The peptide or conjugate according to the invention may also be used in cancer therapy. The present invention thus also relates to any peptide or conjugate according to the invention, or a pharmaceutical composition comprising said peptide or conjugate and a pharmaceutically acceptable vehicle, for use in the treatment of cancer.

The present invention thus also relates to a method for treating cancer in a subject in need thereof, comprising administering to said subject a therapeutically effective amount of any peptide or conjugate according to the invention.

The present invention also relates to a method for in vivo delivery of an entity to an EGFRvIII-expressing cell, comprising administering any conjugate according to the invention to a subject.

The present invention thus also relates to the use of any peptide or conjugate according to the invention, or a pharmaceutical composition comprising said peptide or conjugate and a pharmaceutically acceptable vehicle, in the manufacture of a drug for treating cancer.

The present invention thus also relates to the use of any peptide or conjugate according to the invention, or a pharmaceutical composition comprising said peptide or conjugate and a pharmaceutically acceptable vehicle, for treating cancer.

Preferably, said cancer is an EGFRvIII-expressing cancer, more preferably selected from ovarian cancer, breast cancer and glioblastoma.

By “treatment” is meant an improvement, observed at the clinical or biochemical level, of the subject's disease.

### Diagnosis Methods

The peptide or conjugate according to the invention may also be used for cancer diagnosis, in vitro or in vivo.

The present invention thus also relates to a method for detecting or quantifying EGFRvIII-expressing cells or EGFRvIII-expressing secreted extracellular vesicles (SEV) in a biological sample in vitro or ex vivo, comprising:
a) contacting a biological sample with any peptide or conjugate according to the invention; and
b) detecting or quantifying the peptide or conjugate bound to EGFRvIII-expressing cells or EGFRvIII-expressing SEVs.

The present invention also relates to a method for detecting or quantifying EGFRvIII-expressing cells or EGFRvIII-expressing secreted extracellular vesicles (SEV) in a subject in vivo, comprising:
a) administering any peptide or conjugate according to the invention to a subject; and
b) detecting or quantifying the peptide or conjugate bound to EGFRvIII-expressing cells or EGFRvIII-expressing SEVs in said subject.

### EXAMPLES

The following examples are intended to illustrate the present invention without any limitation.

### Example 1: Identification of a Novel Peptide Ligand for the Cancer-Specific Receptor Mutation EGFRvIII

### 1.1. Materials and Methods

### 1.1.1. Phage Selection

Ph.D.-12™ Phage Display Peptide Library Kit was purchased from New England Biolabs Inc. (Beverly, MA, USA). Biopanning procedures were done according to the manufacturer's instruction with certain modifications. Briefly, a 96-well plate was coated with 150 μL hEGFRvIII (Sino Biological) (in the first two rounds at 100 μg/mL, the third round at 10 μg/mL) overnight at 4° C. Wells were washed with PBS, blocked with blocking buffer (3% BSA in PBS), washed six times with cold PBST (PBS+0.1% [v/v] Tween-20), then incubated with 1010 pfu phage peptide library Ph.D.-12 for 2 hours at RT. Unbound phages were removed by washing 10 times with cold PBST (PBS+0.1% [v/v] Tween-20 in the first round and 0.5% [v/v] in the other two rounds). Bound phages were eluted with 100 μL of 0.2 M Glycine-HCl (pH 2.2), 1 mg/mL BSA and neutralized with 15 μL of 1 M Tris-HCl, PH 9.1. The elution procedure was repeated three times and the final eluate was used for amplification in Escherichia coli ER2738 culture. Recovered phages were subjected for two more rounds of biopanning with hEGFRvIII proteins (Sino Biological). The eluates of each round were titrated by qPCR to enumerate phages and eluate from the third round of screening for NGS sequencing.

Streptavidin was used as a biopanning control with the same condition as the target except for elution. The unbound phages were eluted with 100 μL of 0.1 M Biotin in PBS for 30 minutes.

1.1.2. Phage Titration by qPCR

Quantitative polymerase chain reaction (qPCR) method was used to enumerate M13 phage particles from biopanning. Since each individual phage particle can only contain one copy of genomic DNA (ssDNA), one phage genome is equivalent to one phage particle; by quantifying the number of phage genomes, it is feasible to quantify the number of phages and differentiate between infectious and non-infectious phages with DNase I pre-treatment of the phage samples. During qPCR, fluorescent reporter dyes bind phage genomic DNA through sequence-specific primers during PCR amplification, and the fluorescence signal increases with each round of amplification. When the fluorescence signal reaches the threshold, that round/cycle of amplification is noted as the threshold cycle (Ct). The known concentrations of reference phage DNA are plotted against their Ct values to establish a standard curve. Using the standard curve with the Ct values of DNA samples, the concentrations of phages can be interpolated. Equation (1) was used to convert for M13 phage reference DNA concentrations from fg/μL to genome copies (gc)/μL. Equation (1):

$$M 13 phage DNA concentration in \frac{gc}{ul} = 2 * ( \frac{[ ssDNA ( \frac{g}{ul} ) ]}{[ DNA size ( bp ) * 607.4 + 157.9 )} ) * ( 6.02 * 18 )$$

NOTE: bp: base pair; reference M13 genomic DNA is 7222 bp.

Forward and reverse primers were designed upstream of the library variable region to amplify M13 phage genomic DNA (Table 1).

TABLE 1
Primers used for qPCR.

| Primer name | Primer sequence | SEQ ID NO: |
|---|---|---|
| QPCR-F | ATG CAA GGT ACC TTT | SEQ ID NO: 7 |
| | CTA TTC TCA CTC T | |
| QPCR-R | GTA TGG GAT TTT GCT | SEQ ID NO: 8 |
| | AAA CAA C | |

New dilutions for plasmid standard curves were prepared from stocks for each assay as following: M13KE plasmid with known concentration to 10 ng/μL (107 fg/μL) was diluted ten-fold serial dilution for seven times till reaching 1 fg/μL. 20 μL of each M13 clone (phage) selected from bio-panning against hEGFR were mixed with 1.25 units of DNase I (0.5 μL) and incubated at 37° C. for 10 min and then incubate at 100° C. for 15 min to stop the reaction. The samples were let to cool down slowly at room temperature (RT) before the qPCR reaction.

All qPCRs were performed in 10 μL reactions (8 μl PowerUp™ SYBR™ Green (Bio-Rad), 0.5 μL of 10 μM primer sets and 2 μL template. Standards and unknown samples were assayed in triplicate using the thermocycler cycling conditions set up on the C1000 Touch™ thermal cycler (®BioRad) as follows for a sample volume of 10 μL: one cycle at 50° C. for 2 min, one cycle at 95° C. for 2 min, followed by 40 cycles of (95° C. for 15 s, 60° C. for 1 min). After, a melt curve was running with following settings: one cycle of 95° C. for 15 s, 60° C. for 1 min, and 95° C. for 15 s. The number of M13 phages in the biopanning samples were calculated using the linear regression equation from the standard curve: Y=kX+b (k is the slope of the linear curve; b is the intercept).

### 1.1.3. NGS Library Preparation

The library preparation is done in compliance to illumina NGS library generation protocol. The elution samples from each round of biopanning were subjected to two PCRs. The first PCR adds the barcodes necessary to demultiplex the DNA sequences during data analysis and the second PCR adds the adapter and reading sequences that are important for sequence reading by the iseq100. Phage sample, 20 μL is added with 0.5 μL (1.25 units) DNasel and heated at 37° C. for 10 min to degrade all residual DNA in the mix. Following which, the reaction is heated at 100° C. to stop the DNAse activity and degrade the capsid to release the ssDNA. This ssDNA serves as the template for the first PCR. ssDNA, 10 μL was mixed with 2.5 μL (5 pM) forward and reverse primers, 0.5 mM dNTPs, 10 μL GC enhancer buffer, 10 μL polymerase buffer and 1U of Platinum SuperFi DNA polymerase (thermofisher scientific). The PCR cycle is set to one cycle at 98° C. for 30 sec followed by 25 cycles at 98° C. for 15 sec, 60° C. for 1 min and 72° C. for 1 min. The PCR products are viewed on a 1% agarose gel and any other contaminant bands are discarded by gel purification on a column using a NEB PCR clean up kit. The separate PCR products are pooled and used as a template for the second PCR. The conditions for the 2nd PCR are like the 1st PCR and only 15 cycles were run. Following which, the PCR product (expected size 200 bp) is analyzed on a 1% agarose gel and purified. The concentration of DNA was determined using a dsDNA High Sensitivity Assay Kit (Thermofisher scientific) following the manufacturer's protocol. According to Illumina recommendation, 50 pM of DNA spiked with 10% PhiX control was fed into the flow cell for sequencing by iseq100. The primers used in PCR1 are named as NGS-1, NGS-2, NGS-3, NGS-4 and NGS Rev. The primers used in PCR2 are seq fw and seq rev (primer sequences are provided in (Table 2)).

TABLE 2
Primers used for PCR1 and PCR2 in NGS library preparation.

| Primer | | |
|---|---|---|
| name | Primer sequence | SEQ ID NO: |
| NGS-1 | CTCTTCCGATCTATGCCTTACGGATCCTCATTAATGCAAGCAATT | SEQ ID NO: 9 |
| | CCTTTAGTGGTACCTTTCTATTCTCAC | |
| NGS-2 | CTCTTCCGATCTATGCCTTACGGATCCTCATTAATAAGCGCAATT | SEQ ID NO: 10 |
| | CCTTTAGTGGTACCTTTCTATTCTCAC | |
| NGS-3 | CTCTTCCGATCTATGCCTTACGGATCCTCATTAGCATAAGCAATT | SEQ ID NO: 11 |
| | CCTTTAGTGGTACCTTTCTATTCTCAC | |
| NGS-4 | CTCTTCCGATCTATGCCTTACGGATCCTCATTATATAGCGCAATT | SEQ ID NO: 12 |
| | CCTTTAGTGGTACCTTTCTATTCTCAC | |
| NGS | GTTTCGGCCGAACCACCACC | SEQ ID NO: 13 |
| rev | | |
| seq | AATGATACGGCGACCACCGAGATCTACACGCTTGTCAACACTCT | SEQ ID NO: 14 |
| fw | TTCCCTACACGACGCTCTTCCGATCTATGCCTTACGGATCCTCA | |
| | TTA | |
| seq | CAAGCAGAAGACGGCATACGAGATATCACGACGTGACTGGAGT | SEQ ID NO:15 |
| rev | TCAGACGTGTGCTCTTCCGATCTGTTTCGGCCGAACCACCACC | |

### 1.1.4. NGS Data Analysis

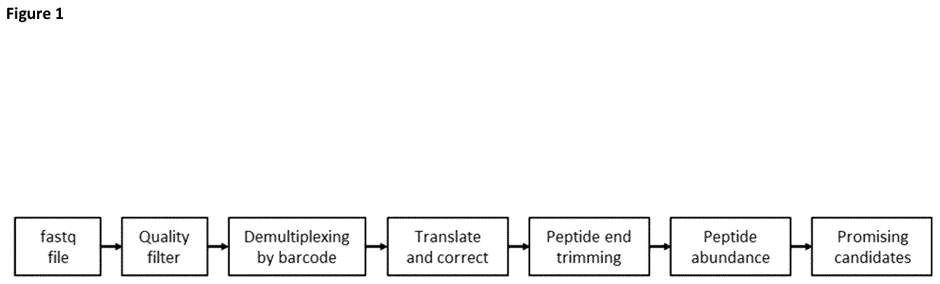

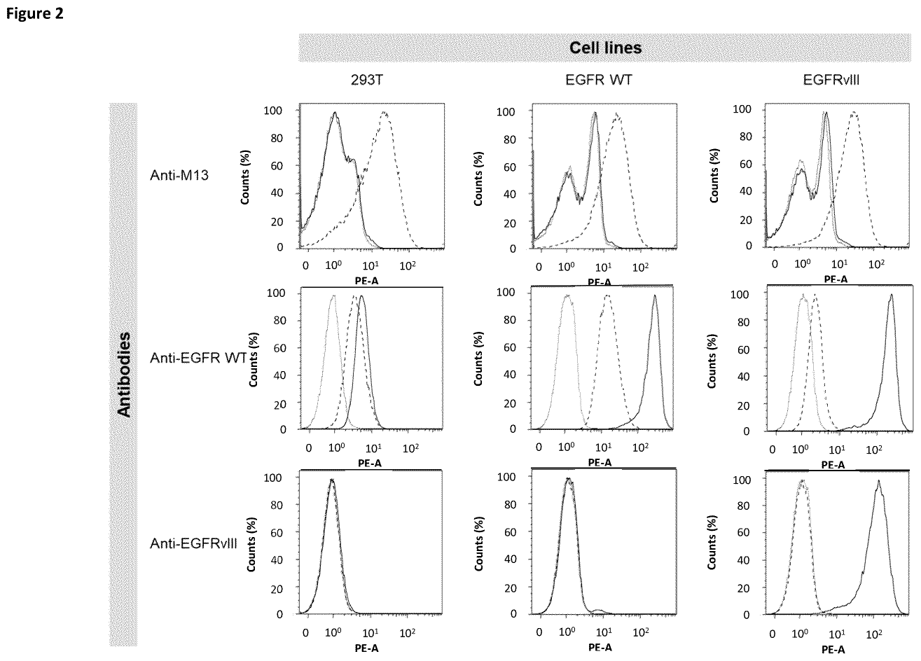

The NGS data analysis is an imperative approach to filter out all the non-specific binders and identify the putative binders. In addition, NGS also allows us to perform deeper quantitative analysis, such as calculating the frequency of amino acids distribution in each position of the peptide sequence, consensus motif and target unrelated peptides. NGS in combination with phage display is used widely these days as it gives us a global idea of how thousands of peptide sequences evolve in subsequent rounds of biopanning unlike Sanger sequencing which only yields sequences of a few clones. This is highly useful as we can follow how the read of a peptide evolves or changes with the rounds of biopanning. The iseq100 generates a fastq file which provides us with the sequence and phred score. There are several programs which are executed sequentially to follow each step of the analysis that is shown in FIG. 1. The overall quality of the NGS sequencing data is first checked using the FASTQC software. The amplicon of interest is a 200 bp including the barcode sequence and the adapter sequence and contains an Eag1 and Kpn1 site. The DNA sequence corresponding to the peptide insert is located in between these restriction sites. The first program extracts only those DNA sequences that contains these restriction sites and has a phred score above 30. The total number of sequences and the corresponding number of reads for each DNA sequence are recorded in a separate CSV file. The second program, demultiplexes the DNA sequences into separate CSV files corresponding to their barcodes that are added in the 1st PCR amplification. Each barcode file corresponds to a specific round of biopanning. The 3rd program is executed on each barcode file where each DNA sequence is translated into the peptide sequence. Any DNA sequence without the peptide insert is discarded. Due to codon degeneracy, different codons can translate into same amino acids, thus after translation identical sequences exist with different reads. A 4th program collates all the identical sequences and their reads into one, thus refining the data. Once the final list of peptides is obtained from each barcode, frequencies of all the peptides are calculated by normalizing the reads. The frequency is the ratio of individual reads to the total number of reads in that specific round. In this way we normalize the difference in the number of reads in the different rounds of biopanning including the background. Post this, the background peptides are filtered out from the peptides in all the rounds. The peptides in the last round of biopanning were sorted according to their enrichment values. The enrichment is the ratio of the frequency of the peptide in the last round to the first round of biopanning. The top 20-30 peptide hits that evolved over the rounds are chosen for further characterization. All the frequency and enrichment calculations are done by command lines written in MS Excel.

The NGS data is further exploited for deeper quantitative analysis. Once the top peptides were sorted, we were also interested in knowing how individual amino acids enrich at different positions in the peptide sequence over subsequent rounds of biopanning. For all the quantitative analysis, in-house programs were written in python to calculate amino acid frequency and consensus sequence/motifs. Similar analysis is done on all the peptides in all the rounds of biopanning. Amino acids that have enriched at specific positions are used to establish a consensus sequence. In addition, we searched for abundantly occurring motifs in the peptides retrieved the last round of biopanning. Presence of a highly abundant motif may indicate that they might be essential in binding and can be further exploited. All the peptides were divided into 3-4 amino acid motifs and each motif was searched in the entire list of peptides and their abundance was calculated. In parallel, the top peptide hits were docked to the target protein to examine their docking scores.

### 1.1.5. Cell Lines and Cell Culture

293T human embryo kidney cells (Sigma; #85120602-1VL), CLTH/EGFR WT cells (Celther; #CL 01010-CLTH) and CLTH/EGFRvIII cells (Celther; #CL 01001-CLTH) were purchased from Sigma and Celther accordingly. CLTH/EGFR WT and CLTH/EGFRvIII cell lines are established from human embryo kidney cells to stably express EGFR WT and EGFRvIII proteins respectively. The three types of cells were maintained in Dulbecco's modified Eagle media (high glucose) supplemented with 10% fetal bovine serum (FBS), 100 U/mL penicillin, 100 mg/mL streptomycin and 1% MEM NEAA. The cells were cultured at 37° C. in a humidified atmosphere containing 5% CO₂.

### 1.1.6. Flow Cytometry

Cell cultures and flow cytometry experiments with phages or peptides were performed by Institut Mondor de Recherche Biomédicale (IMRB U955, France). Prior to be tested with the selected phages or peptides, each cell line was incubated with different commercial antibodies to confirm their phenotype (FIG. 2). A monoclonal anti-EGFR WT antibody (Life technologies) and an anti-EGFRvIII (Millipore) primary antibodies were used in flow cytometry experiments to evaluate the expression of EGFR WT and EGFRvIII receptors on CLTH/EGFR WT cells and CLTH/EGFRvIII cells respectively. Anti-IgG1 conjugated to phycoerythrin (PE) (Miltenyi Biotec) was used as a secondary antibody to read the fluorescence of PE. 293T cells were tested as a negative control. A phage-free control experiment was also performed on each cell type prior to testing phages. Anti-M13 conjugated to biotin (Abcam) was used as a primary antibody, anti-mouse IgG3 kappa monoclonal conjugated to biotin (Abcam) as isotype control and anti-biotin conjugated to PE (Miltenyi Biotec) as secondary antibody.

TABLE 3
list of antibodies and controls used on 293T,
CLTH/EGFR WT and CLTH/EGFRvIII cells.

| | Anti- | Anti- | Anti- |
|---|---|---|---|
| | M13 | hEGFR WT | hEGFRvIII |
| Primary | anti-M13 | anti-EGFR | anti- |
| antibody | conjugated | WT (Life | hEGFRvIII |
| | to biotin (Abcam | Technologies | (Millipore # |
| | #ab17269) | #MA5-13070) | MABS1915) |
| Isotype | mouse IgG3 | mouse IgG1 | mouse IgG1 |
| control | kappa | (Miltenyi | (Miltenyi |
| | monoclonal | Biotec #130- | Biotec #130- |
| | conjugated to biotin | 106-545) | 106-545) |
| | (Abcam #ab18436) | | |
| Secondary | anti-biotin | anti-IgG1 | anti-IgG1 |
| antibody | conjugated to PE | conjugated to PE | conjugated to PE |
| | (Miltenyi Biotec | (Miltenyi Biotec | (Miltenyi Biotec |
| | #130-110-951) | #130-117-098) | #130-117-098) |

On phages. Binding of the selected phages on 293T human embryo kidney, CLTH/EGFR WT and CLTH/EGFRvIII cells was characterized by flow cytometry. Approximately 1×10⁶ 293T cells (expressing endogeneous levels of EGFR WT) were collected, washed with cold PBS and blocked with 2% w/v milk-PBS (MPBS), rotating for 30 min at 4° C. Concurrently, approximately 1010 phages from the rescued library stock were also blocked in MPBS. The blocked phages were then added to the blocked cells and incubated rotating for 1 h at 4° C. After centrifugation at 500 g for 3 min, the supernatant containing non-bound phages was collected. This step allowed the removal of phages that interacted with this EGFR negative cell line (i.e., not an EGFR-specific binding). The supernatant was then used to resuspend transfected cells washed beforehand (CLTH/EGFR WT expressing EGFR WT and CLTH/EGFRvIII expressing EGFRvIII). Treated cells were incubated for 1 h at 4° C., collected by centrifugation and washed two times with PBS at pH 7.4. Cells were stained with Viobility™ 405/520 fixable dye (Miltenyi Biotec) as a viability marker, with anti-M13 conjugated to biotin (Abcam) plus anti-biotin conjugated to PE (phycoerythrin) (Miltenyi Biotec) or anti-biotin conjugated to PE (Miltenyi Biotec) only. Cells were analyzed by flow cytometry on a MACSQuant Analyser 16 (Miltenyi Biotec) using the MACS Quantify software to acquire and process the data. Viobility™ 405/520 fixable dye was excited at 405 nm and fluorescence was collected with the 525/50 nm filter. PE was excited at 488 nm and fluorescence was collected with the 579/34 nm filter. Viable cells were gated, and PE fluorescence intensity was recorded.

On FITC-Ahx-peptides. Flow cytometry analysis was also performed to quantitatively analyze the binding affinity of the synthetized peptides to the different cell types. Briefly, 293T, CLTH/EGFR WT and CLTH/EGFRvIII cells (100 000 cells, 500 μL) were plated in 24-well plates and incubated overnight at 37° C. in a humidified atmosphere containing 5% CO₂ overnight. The next day, cells were treated with FITC-Ahx-peptides using a stock solution in pure DMSO at 5 mM to reach a final concentration of 10 UM (final percentage of DMSO of 0.2%). Plates were incubated for 20 min at RT. Cells were detached from the plates by a PBS fush, transferred to FACS tubes and washed 2 times with 4 mL of PBS. Cells were stained with LIVE/DEAD™ Fixable Near-IR Dead Cell Stain Kit (Thermofisher) as a viability marker. Cells were analyzed by flow cytometry on a MACSQuant Analyser 16 (Miltenyi Biotec) using the MACS Quantify software to acquire and process the data. LIVE/DEAD™ Fixable dye was excited at 640 nm and fluorescence was collected with the 785/62 nm filter. FITC was excited at 488 nm and fluorescence was collected with the 525/50 nm filter. Viable cells were gated, FITC fluorescence intensity was recorded and the mean fluorescence intensity (MFI) was measured. MFI values were then normalized by substracting MFI from blank cells and dividing by the correction factor determined using a spectrofluorimeter.

### 1.1.7. FITC-Ahx-Peptide Synthesis

The candidate peptides were synthesized by ProteoGenix (France) using standard solid-phase Fmoc chemistry. FITC was conjugated to the N-terminus of each candidate peptide with a 6-aminohexanoic acid (Ahx) linker. The products were purified to a minimum purity of 95% by high-performance liquid chromatography (HPLC) and isolated by lyophilization. The sequence and structure of each peptide were characterized by mass spectrometry, and the purity of the peptides was determined by analytical HPLC.

### 1.1.8. Correction Factor of FITC-Ahx-Peptides

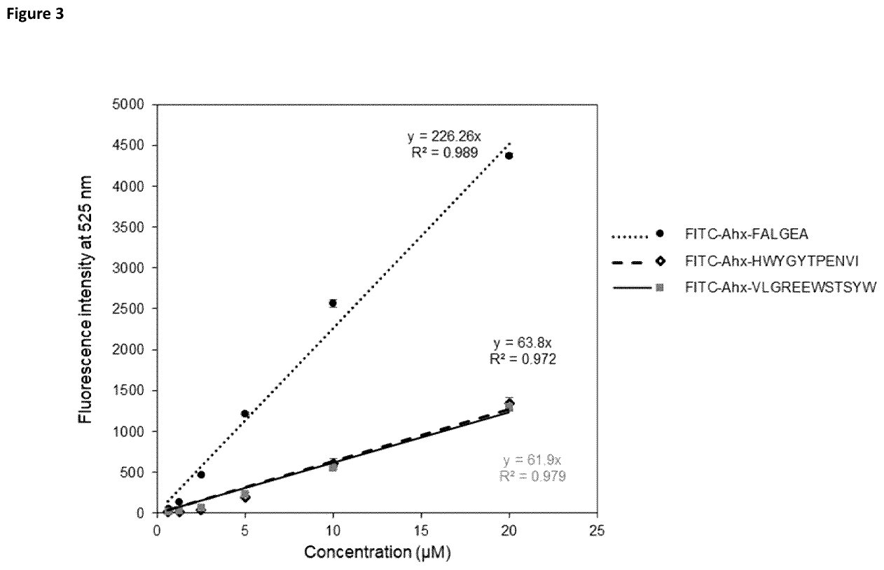

Since FITC fluorescence can be impacted by the nature of the amino acids in the peptide sequence and since the conjugation rate of FITC in the peptide synthesis can fluctuate, it was needed to measure a correction factor according to the fluorescence intensity of each peptide. As flow cytometry cannot analyze fluorescence of small molecules, a spectrofluorimeter was used in similar conditions (excitation and emission) to the flow cytometry conditions. Fluorescence intensities at 525 nm of the FITC-Ahx-peptides in PBS were measured using a Varioskan plate reader (Thermo Scientific) and the Skanlt Software 5.0. Samples with concentrations between at 0.625, 1.25, 2.5, 5, 10 and 20 μM were prepared in triplicates using stock solutions of peptides in DMSO at 5 mM. Samples (100 μL) were analyzed in a 96-well plate using the following conditions: temperature of 25° C., excitation wavelength at 490 nm, excitation bandwidth of 5 nm, emission wavelength at 525 nm, by step of 1 nm, measurement time 100 ms, and upper lens. Linear regressions were determined for each peptide. The correction factor was determined by dividing each slope by the arbitrary value 100 and was used to normalize the MFI obtained by flow cytometry. Results are shown in FIG. 3 and Table 4.

TABLE 4
Slope, R² and correction factor for each peptide.

| | Peptide | | | |
|---|---|---|---|---|
| | FITC- | FITC-Ahx- | FITC-Ahx- | Correc- |
| | Ahx- | YHWYGY | VLGREE | tion |
| | FALGEA | TPENVI | WSTSYW | to |
| Slope | 226.3 | 63.8 | 61.9 | 100.0 |
| R² | 0.989 | 0.972 | 0.979 | |
| Correction | 2.26 | 0.64 | 0.62 | 1.00 |
| Factor | | | | |

### 1.1.9. Computational Protein-Peptide Docking Study

The wild type EGFR protein was downloaded from RCSB-Protein Data Bank with ID: 3NJP (Extracellular and Transmembrane Domain Interfaces in Epidermal Growth Factor Receptor as initial structure) (Lu et al., 2010). The protein was further prepared for computational usage in Chimera software without disturbing the back bone of the protein (Pettersen et al., 2004). The protein is a dimer and has two chains, therefore truncation was executed on both the chains. The region from VCQGT to CVKKCPR was sliced and saved as separate PDB file. The N-terminal LEEKKG part was retained by building the amino acids. The truncated protein dimer was relaxed by energy minimization using Swiss PDB viewer by applying GROMOS forcefield (Guex et al., 1999; Gunsteren, 1996). The conformation with local minima was considered for peptide docking in further docking studies. The active site of the protein in dimer form of EGFRvIII was predicted using CastP server tool (Tian et al., 2018). The peptides were built as linear fragments to give high flexibility during docking. Using HPEPDOCK server, (Zhou et al., 2021) global docking was performed and 100 conformations per each peptide were generated. Similar methodology will also be followed to the peptide that shows affinity towards EGFRvIII and EGFR-WT for cross verification of peptide specificity. The top scored ligands as well as the ligands that show affinity towards the identified active sites were sorted. The number of conformations of peptide that shows affinity to a protein is a parameter for peptide specificity. The conformation that exists at the active site were ranked based on score value. The model lowest binding affinity value was considered as best conformation for further analysis and interaction data with the target protein.

### 1.2. Results

### 1.2.1. Enrichment of EGFRvIII-Binding Peptide-Phages

Three rounds of affinity selection were performed to select the specific EGFRvIII-binding phages from the Ph.D.-12™ Phage Display Peptide Library using purified and active extracellular domain for EGFRvIII protein. qPCR was used to accurately enumerate phage particles during the 3 rounds of affinity selection to quantify phages that bind to the substrate. High phage enrichment rate was observed after each round of selection, which indicates successful affinity selection of biopanned phage for the target.

### 1.2.2. Diversity of Phage-Selected Peptides

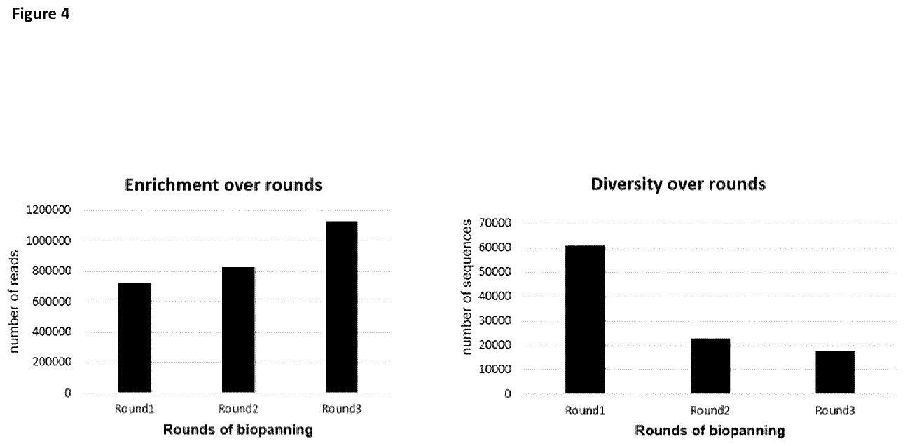

The eluates from all the rounds of biopanning were sequenced by illumina following PCR with the adaptor and reading primer sequences. Results show (FIG. 4) that the total number of reads increases over the rounds while the diversity of the peptides decreases. These results are an indication that target specific peptides were recovered.

### 1.2.3. Evaluation of the Specific Binding of the Selected Phage Displayed Peptides

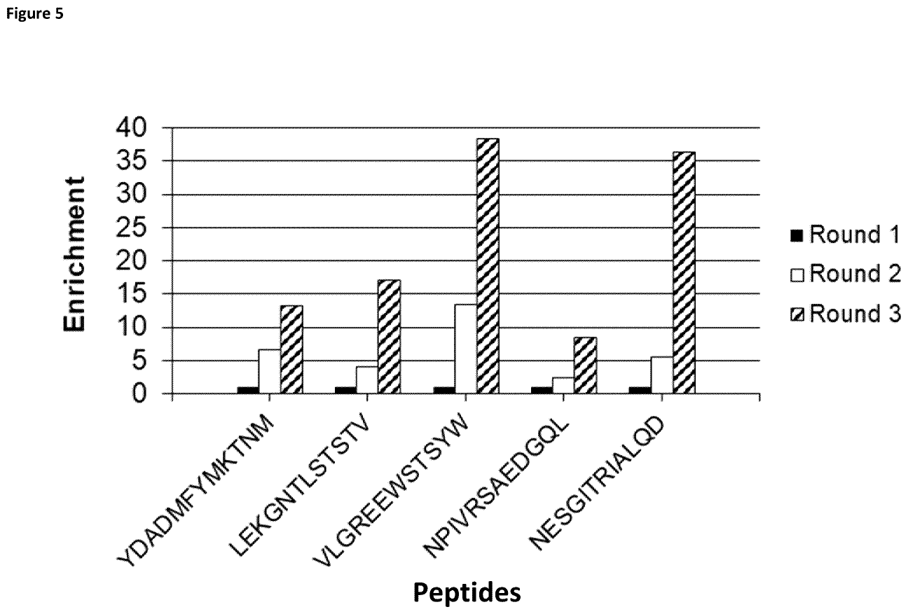

The binding of phages displaying 5 peptides selected from the NGS data analysis at their surface was evaluated. The criteria used for the selection was the peptides enrichment over the rounds of biopanning and the frequency of these peptides if present in the background should be below 0.002. NPIVRSAEDGQL (SEQ ID NO: 17) corresponds to negative hit because it didn't show any significant enrichment on NGS (FIG. 5).

To that end, the 5 peptides displayed on the phage were directly tested by flow cytometry for their specificity to stable cell lines expressing hEGFRvIII and hEGFR WT. The parental cell line 293T was used as a negative control as it expresses very low levels of endogenous EGFR WT, does not express hEGFRvIII and is a non-cancerous cell line.

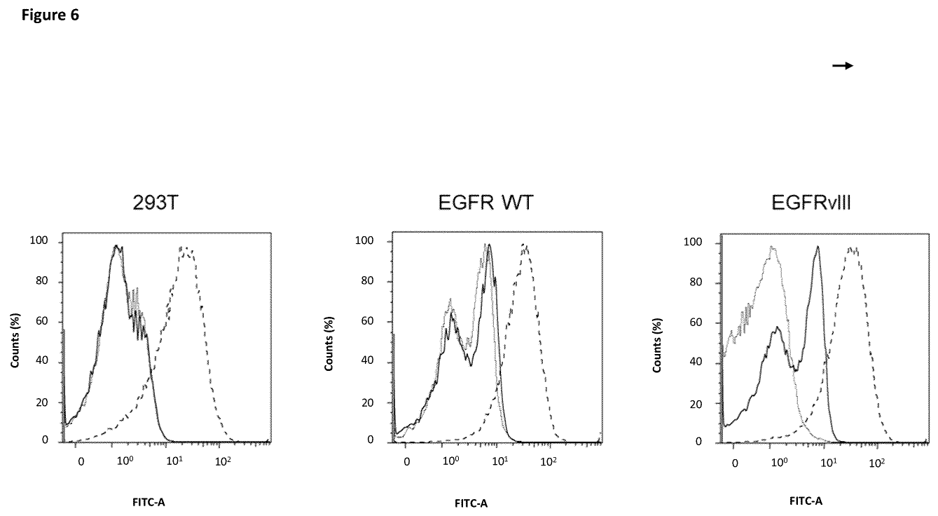

Among tested peptides, only VLGREEWSTSYW-M13 phage displayed peptide showed specific binding to hEGFRvIII, giving confidence that the phage was binding to the cell-surface exposed region of their respective target antigen. The fluorescence intensity for the VLGREEWSTSYW-M13 phage displayed peptide was strong in hEGFRvIII cells (see arrow FIG. 6), while its fluorescence intensity was weak in hEGFR WT cells and in the negative control cell line (293T) (FIG. 6). Of note, the dashed line observed on FIG. 6, which corresponds to the isotype labelling displays unspecific binding to the anti M-13 antibody. Said unspecific binding was already observed in FIG. 2 where labelling is observed for all 3 cell types in the M-13 condition. In contrast, other sequences such as LEKGNTLSTSTV (SEQ ID NO: 16) and NESGITRIALQD (SEQ ID NO: 18), selected from the NGS analysis, did not show specific binding to hEGFRvIII cells (data not shown).

### 1.2.4. Validation of the Specific Binding Affinity of the Peptide to EGFRvIII by Flow Cytometry

To confirm the results, a synthetic VLGREEWSTSYW (SEQ ID NO: 4) peptide bound to a fluorescent marker was synthetized and the binding specificity towards EGFRvIII versus EGFR WT was evaluated in vitro. To this end, the VLGREEWSTSYW (SEQ ID NO: 4) peptide was synthesized and conjugated with a fluorescent marker fluorescein isothiocyanate (FITC) at the N-terminus using a 6-aminohexanoic acid (Ahx) as a linker. Two peptides from the literature were also synthesized and linked to the FITC-Ahx tag. These were used as controls to evaluate the binding efficiency and specificity of the presently identified peptides. The first peptide ‘FALGEA’ (SEQ ID NO:5) that should be able to target both EGFR WT and EGFRvIII (Denholt et al, 2009, Denholt et al, 2011; Mao et al, 2017) while the second peptide ‘YHWYGYTPENVI’ (SEQ ID NO: 6) is only recognized as a EGFR WT binder (Hossein et al, 2009) and to our knowledge, its potential ability to bind EGFRvIII is unknown.

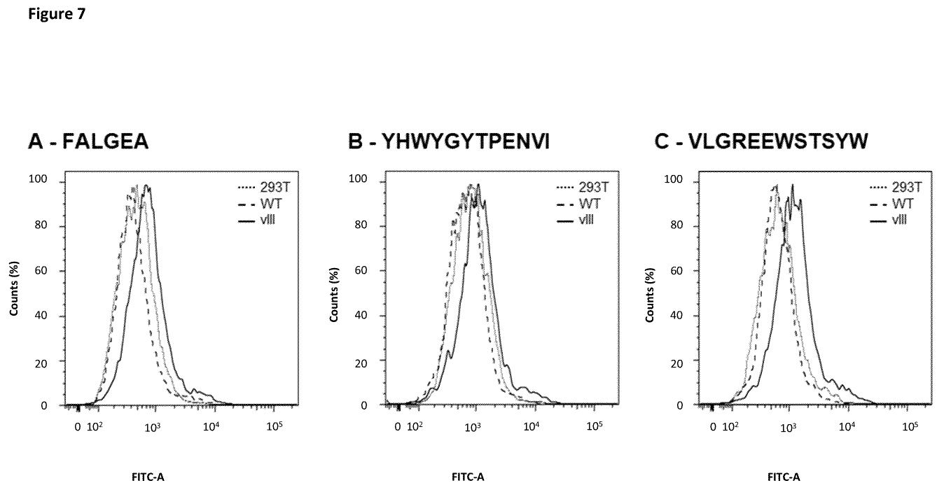

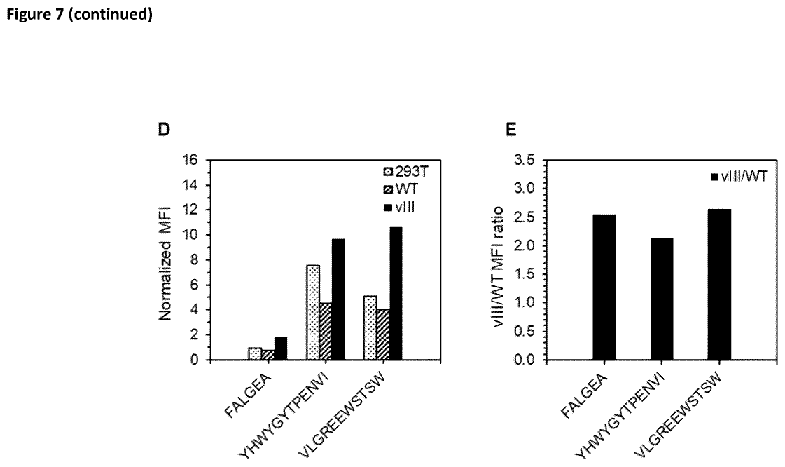

The 293T cells (expressing low endogenous level of EGFR WT), EGFR WT cells (expressing EGFR WT) and EGFRvIII cells (expressing EGFRvIII), as described in paragraph 1.1.5. above, were incubated with 10 UM of the 3 FITC-Ahx-peptides for 20 min at RT. FITC fluorescence intensity of the cells was measured by flow cytometry. Mean fluorescence intensities (MFI) were determined as well as the ratio of MFI between EGFRvIII and EGFR WT (EGFRvIII/EGFR WT) to assess EGFRvIII-binding specificity. The results are shown in FIG. 7 and Table 5. A shift of fluorescence was observed for VLGREEWSTSYW (SEQ ID NO: 4) peptide (according to the invention) on EGFRvIII compared to 293T and EGFR WT cell lines (FIG. 7C). The observed shift is higher than for the control peptides (FIGS. 7A, and 7B).

TABLE 5
Normalized MFI values and vill/WT ratios
for each peptide and each cell line
obtained by flow cytometry.

| | | | | Normalized |
|---|---|---|---|---|
| | Normalized MFI | | | MFI ratio |
| Sequence | 293T | WT | vIII | VIII/WT |
| FITC-Ahx-FALGEA- | 0.9 | 0.7 | 1.8 | 2.6 |
| CONH₂ | | | | |
| FITC-Ahx-YHWYGYT | 7.6 | 4.5 | 9.7 | 2.1 |
| PENVI-CONH₂ | | | | |
| FITC-Ahx-VLGREEW | 5.1 | 4.0 | 10.7 | 2.7 |
| STSW-CONH₂ | | | | |

In particular, results show that, in the tested conditions, the specificity between EGFRvIII and EGFR WT is better for the VLGREEWSTSYW (SEQ ID NO:4) peptide (according to the invention) compared to the control peptide YHWYGYTPENVI (SEQ ID NO:6) (FIG. 7E).

Regarding the normalized MFI data, they show that the binding to EGFRvIII cells was much higher for the VLGREEWSTSYW (SEQ ID NO:4) peptide (according to the invention) compared to FALGEA. Furthermore, the non-specific interactions (293T cell binding) were minimized for the VLGREEWSTSYW (SEQ ID NO:4) peptide (according to the invention) compared to the control peptide YHWYGYTPENVI (SEQ ID NO:6) (FIG. 7D).

Altogether the data show that the VLGREEWSTSYW (SEQ ID NO:4) peptide (according to the invention) binds to EGFRvIII cells with high efficiency and good specificity compared to EGFR WT cells and with less unspecific interactions (293T cells). It can be concluded that the VLGREEWSTSYW (SEQ ID NO:4) peptide (according to the invention) is better for specific targeting of EGFRvIII-expressing cancer cells than peptides known from the art.

### 1.2.5. Validation of Peptide Binding by Computational Docking

As an alternative approach, the binding affinity of the peptide to EGFRvIII was evaluated by computational docking. Computational docking is a common and robust approach to predict protein-protein/peptide interactions and expediates the peptide discovery cycle. Various parameters such as binding affinity, specific residues involved in interaction and number of stable conformations can be obtained rapidly. These data are useful as a basis of peptides screening to be further exploited for experimental validation. As the 3D model of EGFRvIII is not available, the protein structure was predicted by homology modelling. Modelling and subsequent energy minimization resulted in the conformation to be used for active site search. The amino acids 6-273 in EGFR wild type protein were removed on both the chains and the cross connected di-sulfide bridges were reduced in number. The structure was found stable by forming two disulfide interactions in each chain from Lys 304 to Asp 279. The minimization protocol of 100 steepest descent steps at 0.02 Å cut-off, followed by 10 conjugate gradient steps with same cut-off range resulted in a local minimum structure which had a root-mean-square deviation RMSD of 0.9 Å. The binding sites in the protein were explored using CastP server tool. The tool is based on theoretical and algorithmic results of the computational geometry of the proteins. Five hydrophobic pockets were found present in the dimer, towards the N-terminal.

Docking was carried out using HPEPDOCK server tool by considering the active sites as a search space. The docking scores are represented in Table 6. Peptides that show good binding affinity and show the ability to form maximum number of complexes by interacting with any of the active sites of the EGFRvIII protein are considered as putative hits. The results show that the peptide VLGREEWSTSYW (SEQ ID NO: 4) has good affinity compared to the other selected peptides. Notably, LEKGNTLSSTSV (SEQ ID NO: 16) and NESGITRIALQD (SEQ ID NO: 18) have lower docking scores compared to VLGREEWSTSYW (SEQ ID NO: 4). Hence the results of computational docking study are consistent with the flow cytometry experiments.

TABLE 6
Peptide docking result interpretation.

| | Docking | Number | |
|---|---|---|---|
| Peptide | score | of stable | SEQ ID |
| Sequence | (kcal/mol) | conformations | NO |
| VLGREEWSTSYW | −186.5 | 23 | SEQ ID |
| (according to | | | NO: 4 |
| the invention) | | | |
| LEKGNTLSTSTV | −140.5 | 25 | SEQ ID |
| | | | NO: 16 |
| NPIVRSAEDGQL | −148.7 | 38 | SEQ ID |
| | | | NO: 17 |
| NESGITRIALQD | −150.0 | 13 | SEQ ID |
| | | | NO: 18 |
| YDADMFYMKTNM | No | 7 | SEQ ID |
| | interaction | | NO: 19 |

### 1.3. Conclusion

Cancer cells often display high numbers of certain cell surface molecules, such as tumor associated antigens or specific receptors, that rarely occur in normal tissues and represent potential targets for tumor diagnosis and treatment. EGFRvIII is a receptor present in a number of human malignancies such as ovarian cancer, breast cancer and glioblastomas, and was never identified in normal tissues. The screening and identification of peptides that specifically bind to EGFRvIII is an important outcome to promote the development of novel probes for cancer detection and therapy. For this purpose, we utilized a 12-mer phage display peptide library to obtain peptides that specifically bind to EGFRvIII.

In this study, 5 peptides from the third round of biopanning have been selected based on the top hits identified by high-throughput sequencing analysis according to their enrichment over rounds. Subsequently, the 5 selected phage-displayed peptides were cloned, sequenced and tested using flow cytometry to confirm their specific binding to EGFRvIII cells. Among the 5 selected phage-displayed peptides, VLGREEWSTSYW (SEQ ID NO: 4) appeared to show specific binding to the EGFRvIII cells compared to the EGFR WT and the parental cells, giving confidence that the phage was binding to the cell-surface exposed region of its respective target antigen. It was not the case for the other 4 tested phage-displayed peptides. Therefore, VLGREEWSTSYW (SEQ ID NO: 4) was selected for further investigation. A multiple sequence alignment showed that the sequence did not exhibit homology to the sequences of any characterized proteins in various protein databases. This finding demonstrates that VLGREEWSTSYW (SEQ ID NO: 4) is a novel peptide that may mimic a complex epitope, which may explain why this novel peptide is not present in any database.

The docking results suggest that the identified peptide, binds to the human cancer-specific epidermal growth factor tyrosine kinase receptor mutation variant III (EGFRvIII) and may therefore be used to target specifically cancer cells expressing EGFRvIII.

The EGFRvIII extracellular domain structure was predicted by homology modelling. The secondary structures of the peptides were predicted and docked on the active sites of EGFRvIII. Interestingly, we observed that the VLGREEWSTSYW (SEQ ID NO: 4) peptide showed high binding affinity towards EGFRvIII compared to all the other peptides tested. This corroborated our experimental data and further guarantees the credibility of the VLGREEWSTSYW (SEQ ID NO: 4) peptide being an EGFRvIII specific peptide.

In conclusion, the present study has allowed the identification of a novel peptide VLGREEWSTSYW (SEQ ID NO: 4) which has good binding efficiency and good binding specificity towards the EGFRvIII. Vector-peptides have many advantages over the more frequently-used vector-antibodies, this is notably due to their smaller size, which facilitates their engineering, but also to their higher cell penetration ability and lower immunogenicity. Hence, the VLGREEWSTSYW (SEQ ID NO: 4) peptide can be used as a new targeting agent for the transfer of drugs (such as chemotherapeutics), or molecules of interest (such as cell markers) directly to cancer cells into tumors. These promising results open the door for the development of new vectors for the treatment or the diagnosis of cancers, in particular cancers expressing EGFRvIII.

### Example 2: Consensus Sequence Identification

The aim of this study is to identify and develop a new consensus sequence based on the peptide VLGREEWSTSYW (SEQ ID NO: 4; as described in Example 1) using in silico methods such as computational docking studies.

### 2.1. Strategy 1: Molecular Docking (Docking, Affinity Maturation, Molecular Dynamics)

### 2.1.1. Site Specificity (Docking)

The thermodynamic affinity of the peptide at the proteins active site is a crucial information to study the system. The most accurate assumption of the placement of the peptide can be attained by a most rigorous and highly efficient docking study.

Blind docking was carried out using HPEPDOCK server tool by considering whole protein as search space. The docking algorithm will generate approximately 1000 conformations of any peptide. The best conformation that shows highest binding affinity will be the docked complex of further study.

The results show that the peptide VLGREEWSTSYW (SEQ ID NO: 4) has good affinity. Simultaneously, the docking study was performed for the top peptide sequence with the wild type EGFR protein. Interestingly the result confirmed that VLGREEWSTSYW peptide shows less affinity towards wild type EGFR, and bind more tightly to the EGFRvIII.

The peptide that shows good binding affinity and which enables to form maximum number of complexes by interacting with any of the EGFR active site is considered as the best peptide.

### Hydrophobic Cavities

The binding sites in the protein are explored using CastP server tool. The tool is based on theoretical and algorithmic results of the computational geometry of the proteins. There are 5 hydrophobic pockets present in the dimer, towards the N-terminal. The binding sites of both the wild type and vIII variant proteins are predicted using CastP. The server gives an idea of the hydrophobic area present in both the proteins. In the wild type, this area is 1388.2 Å² (square angstroms), while in EGFRvIII it is 183.4 Å². The site available with 183.4 Å² is our potential active site deducted from in silico study.

The hydrophobic moment will generally point to the interior of the protein. The side chains of hydrophobic residues are oriented towards the interior of a protein. The hydrophobic moment of a residue is proportional to the hydrophobicity of the residue and whose direction is dependent on the side chain orientation. The hydrophobic moments of side chains of residues forming a secondary structure element can give indication of the preferred orientation of the region of protein.

### Fate of VLGREEWSTSYW Peptide (Known to be Near Native) in the Active Site Region

The pattern of binding of VLGREEWSTSYW (SEQ ID NO: 4) peptide shows that it spans from Site 1 to Site 2, where 2 of 3 best peptide conformations fit in. The hydrophobic N-terminal is fixed in the Site 1. The other 3rd conformation still resides near the binding site region covering the site 2.

The residues Arg 4, Ser 8, and Trp 7 show hydrogen bonds in nature inside the complex. Since the area of search is confined, the best pose of the docking study can be used for further studies such as affinity maturation and molecular dynamics.

TABLE 7
Inter-molecular interactions between
EGFRvIII with VLGREEWSTSYW peptide.

| S. No | From | To | Distance (Å) | Type |
|---|---|---|---|---|
| 1. | ARG 310 | GLU 6 | 4.863 | Electrostatic |
| 2. | LYS 375 | GLU 6 | 5.073 | Electrostatic |
| 3. | ARG 4 | GLU 376 | 5.304 | Electrostatic |
| 4. | ARG 4 | GLU 376 | 3.197 | Electrostatic |
| 5. | LYS 375 | SER 8 | 2.527 | Hydrogen bond |
| 6. | ARG 4 | THR 339 | 2.914 | Hydrogen bond |
| 7. | ARG 4 | THR 339 | 2.719 | Hydrogen bond |
| 8. | ARG 4 | THR 339 | 2.774 | Hydrogen bond |
| 9. | LYS 336 | TRP 12 | 4.450 | Electrostatic |
| 10. | SER 428 | TRP 7 | 4.041 | Hydrogen bond |
| 11. | LYS 336 | TRP 12 | 3.904 | Hydrophobic |
| 12. | ARG 310 | VAL 1 | 4.211 | Hydrophobic |
| 13. | TRP 7 | ARG 509 | 4.164 | Hydrophobic |
| 14. | TRP 7 | ARG 427 | 5.343 | Hydrophobic |
| 15. | TRP 7 | ARG 509 | 4.643 | Hydrophobic |
| 16. | TRP 12 | LYS 336 | 5.125 | Hydrophobic |

Table 7 shows the inter-molecular interactions in the protein-peptide complex, that explains the peptide binds and holds strongly in the hydrophobic cavity of the EGFRvIII.

In conclusion, it can be considered that the positions, 1 (V), 4 (R), 6, 7, 8 (EWS) and 12 (W) are important for the binding according to the molecular docking analysis.

TABLE 8

| | |
|---|---|
| Stage 1: Docking | VLGREEWSTSYW |
| | (SEQ ID NO: 4) |

### 2.1.2. Affinity Maturation

To maximize the probability of identifying mutations that would modify protein-peptide binding affinity, in silico approach was used to perform the mutagenesis with Schrödinger Biologics Suite 2020-3. The designable residues were limited to a sequence space in the variable region while avoiding framework residues.

Decrease in value below 0 (ΔΔG becomes positive) hints the attaining of stability (favorable) after mutation of Parent residue to other residues of interest. The table gives the affinity values where light grey represent attaining slightly better stability and dark grey shows considerable affinity after mutation. Results are based on 95 consecutive mutations of each residue into the target amino acids.

TABLE 9
Affinity maturation of the VLGREEWSTSYW (SEQ ID NO: 4) peptide against EGFRvIII.

| | | 1 | 2 | 3 | 4 | 5 | 6 | 7 | 8 | 9 | 10 | 11 | 12 |
|---|---|---|---|---|---|---|---|---|---|---|---|---|---|
| RES | | VAL | LEU | GLY | ARG | GLU | GLU | TRP | SER | THR | SER | TYR | TRP |
| ALA | NP | 6.54 | 12.89 | −2.03 | 20.00 | −1.19 | 8.20 | 14.44 | 1.36 | 0.13 | −0.19 | 1.47 | 10.12 |
| ASN | +ve | 2.60 | 6.920 | −2.81 | 19.84 | −4.67 | 3.44 | 10.48 | −4.91 | 0.05 | −1.57 | 1.62 | 8.99 |
| ASP | −Ve | 3.19 | 12.51 | 2.21 | 16.80 | 1.4 | 1.80 | 21.77 | 1.7 | 0.22 | 3.81 | 2.13 | 11.28 |
| CYS | P, | 4.23 | 11.07 | −2.3 | 18.77 | −2.67 | 5.56 | 11.70 | −0.25 | −0.35 | −1.06 | 1.18 | 9.35 |
| | Nu | | | | | | | | | | | | |
| LYS | +ve | 4.19 | 17.25 | −2.05 | 10.15 | −8.02 | 9.83 | 5.90 | −6.75 | 0.07 | 0.00 | 1.96 | 5.70 |
| PHE | NP, | −10.4 | −5.74 | −6.85 | 13.56 | −1.57 | 0.25 | 6.15 | −1.14 | −9.42 | −4.56 | 0.42 | 0.78 |
| | Ar | | | | | | | | | | | | |
| PRO | P, | 1.10 | 11.24 | −6.65 | 18.23 | −0.97 | 6.48 | 17.05 | 0.02 | −0.32 | −2.32 | 0.98 | 9.26 |
| | Nu | | | | | | | | | | | | |
| SER | P, | 4.33 | 13.47 | −1.36 | 20.30 | −3.7 | 4.81 | 10.17 | 0.00 | 0.04 | −4.5 | 1.63 | 10.35 |
| | Nu | | | | | | | | | | | | |

In conclusion, it can be considered that the positions 4 (R), 6, 7 (EW), 11 and 12 (YW) are important for the binding according to the affinity maturation study.

TABLE 10

| | |
|---|---|
| Stage 2: | |
| Affinity | VLGREEWSTSYW |
| maturation | (SEQ ID NO: 4) |

### 2.1.3. Molecular Dynamics

The systems were minimized with 10000 steepest descent steps followed by gradual heating from 0 to 300 K, under NVT ensemble (canonical ensemble, amount of substance (N), volume (V) and temperature (T) are conserved). The systems were thermally relaxed before the production run using Nose-Hoover Chain thermostat method for 2 ns and pressure relaxation with Martyna-Tobias-Klein barostat method. Finally, 10 ns production run under NPT ensemble (isothermal-isobaric ensemble, amount of substance (N), pressure (P) and temperature (T) are conserved) was carried out using a cut-off distance of 9 Å for non-bonded interactions.

Coordinates were saved at each 10 ps to generate trajectories of 1000 frames each. After reaching the equilibrium, molecular dynamics simulations were run during 10 ns, and the snapshot configurations were saved at every 10 ps interval. The system came to equilibrium at 1.2 ns with 250 snapshots. The RMSF (root mean square fluctuation) of both protein and peptide was studied from step 200 to 230 when the system showed equilibrium.

TABLE 11
Molecular dynamics analysis of residue participation
at stable peptide-protein complex.

| | | | | | | | | | | | | |
|---|---|---|---|---|---|---|---|---|---|---|---|---|
| MD time step | | | | | | | | | | | | |
| 200-230 | | | | | | | | | | | | |
| (0.96 ns to | | | | | | | | | | | | |
| 1.14 ns) | V | L | G | R | E | E | W | S | T | S | Y | W |

| | | | | | | | |
|---|---|---|---|---|---|---|---|
| 230 | | 4 | 6 | 7 | 8 | | 12 |
| 229 | | 4 | 6 | 7 | 8 | | 12 |
| 228 | | 4 | 6 | 7 | | | 12 |
| 227 (best conf) | 1 | 4 | 6 | 7 | 8 | | 12 |
| 226 | | 4 | 6 | 7 | 8 | | 12 |
| 225 | | 4 | 6 | 7 | 8 | | 12 |
| 224 | | 4 | | 7 | 8 | | 12 |
| 223 | | 4 | | 7 | 8 | | 12 |
| 222 | | 4 | | 7 | 8 | | 12 |
| 221 | | 4 | | 7 | 8 | | 12 |
| 220 | | 4 | | 7 | 8 | | 12 |
| 219 | | 4 | 6 | 7 | 8 | | 12 |
| 218 | | 4 | 6 | 7 | 8 | | 12 |
| 217 | | 4 | 6 | 7 | 8 | | 12 |
| 216 | | 4 | | 7 | 8 | | 12 |
| 215 | | 4 | 6 | 7 | 8 | | 12 |
| 212 | | 4 | | 7 | 8 | | 12 |
| 213 | | | | 7 | 8 | | 12 |
| 212 | | 4 | | 7 | 8 | | 12 |
| 211 | | 4 | | 7 | 8 | | 12 |
| 210 | | | 6 | 7 | 8 | | 12 |
| 209 | | 4 | | 7 | 8 | 10 | 12 |
| 208 | | | 6 | 7 | 8 | | 12 |
| 207 | | 4 | | 7 | 8 | | 12 |
| 206 | | | | 7 | 8 | | 12 |
| 205 | | | | 7 | 8 | | 12 |
| 20 | | | | 7 | 8 | | 12 |
| 203 | | | | 7 | 8 | | 12 |
| 202 | | | 6 | 7 | | | 12 |
| 201 | | | 6 | 7 | | | 12 |
| 200 | | | 6 | 7 | | | 12 |

The root mean square fluctuation (RMSF) of both protein and peptide was studied.

The time steps at which the protein and the peptide came to equilibrium were from 200 to 230, 227^{th} step is the equilibrium state that defines stable protein complex.

In conclusion, it can be considered that the positions, 1 (V), 4 (R), 6, 7, 8 (EWS) and 12 (W) are important for the binding according to the Molecular dynamics analysis.

TABLE 12

| | |
|---|---|
| Stage 3: | VLGREEWSTSYW |
| Molecular | (SEQ ID NO: 4) |
| Dynamics | |

### 2.2. Strategy 2: Re-Docking of Mutant Peptides (Alanine Mutation Docking)

### 2.2.1. Alanine Mutant Docking Study

A change in binding free energy up on mutation of a selected residue to alanine should correspond to the contribution of that residue to the binding affinity. Negative numbers in ΔΔG subtotal means highly unfavorable substitutions. In contrast, positive ΔΔG subtotal indicates the preference for the alanine residue at the mutated position. The difference of minimum+/−4.8 kcal/mol is considered as a significant change in ΔΔG.

The generated peptide conformations have undergone only local changes to the peptide structure, which ensures the changes did not affect the total binding pattern to the protein, and also the shape of the complex. These residues exhibit hydrophobic interactions with the nonpolar groups that are lining in the active site cleft of the protein.

TABLE 13
Alanine scan study with EGFRvIII.

| | | Hydrophobic | Hydrophilic | | | |
|---|---|---|---|---|---|---|
| | | moment | moment | | | Effect |
| | | residues in | residues in | Dock Score | ΔΔG | upon |
| S. No | Peptide | Bold | Bold | (kcal/mol) | (kcal/mol) | mutation |
| REF | VLGREEWSTSYW | 1, 2 | 4, 5, 6 | −186.5 | — | — |
| | SEQ ID NO: 4 | | | | | |
| 1. | ALGREEWSTSYW | 1, 2 | 4, 5, 6 | −182.5 | −4.0 | No |
| | SEQ ID NO: 20 | | | | | change |
| 2. | VAGREEWSTSYW | 1, 2 | 4, 5, 6 | −192.5 | 6.0 | Stable |
| | SEQ ID NO: 21 | | | | | |
| 3. | VLAREEWSTSYW | 1, 2, 3 | 4,5, 6 | −171.4 | −15.1 | Instable |
| | SEQ ID NO: 22 | | | | | |
| 4. | VLGAEEWSTSYW | 1, 2, 4 | 5, 6 | −190.2 | 3.7 | No |
| | SEQ ID NO: 23 | | | | | change |
| 5. | VLGRAEWSTSYW | 1,2,5 | 4, 6 | −199.1 | 12.6 | Stable |
| | SEQ ID NO: 24 | | | | | |
| 6. | VLGREAWSTSYW | 1, 2, 6 | 4, 5 | −194.1 | 7.6 | Stable |
| | SEQ ID NO: 25 | | | | | |
| 7. | VLGREEASTSYW | 1, 2, 7 | 4, 5, 6 | −176.4 | −10.1 | Instable |
| | SEQ ID NO: 26 | | | | | |
| 8. | VLGREEWATSYW | 1, 2, 8 | 4, 5, 6 | −182.7 | −3.8 | No |
| | SEQ ID NO: 27 | | | | | change |
| 9. | VLGREEWSASYW | 1, 2, 9 | 4, 5, 6 | −181.7 | −4.8 | Instable |
| | SEQ ID NO: 28 | | | | | |
| 10. | VLGREEWSTAYW | 1, 2, 10 | 4, 5, 6 | −169.2 | −17.3 | Instable |
| | SEQ ID NO: 29 | | | | | |
| 11. | VLGREEWSTSAW | 1, 2, 11 | 4, 5, 6 | −167.3 | −19.2 | Instable |
| | SEQ ID NO: 30 | | | | | |
| 12. | VLGREEWSTSYA | 1, 2, 12 | 4, 5, 6 | −167.1 | −19.4 | Instable |
| | SEQ ID NO: 31 | | | | | |
| 13. | VLAREEAATSYW | 1, 2, 3, 7, 8 | 4, 5, 6 | −189.0 | 2.5 | No |
| | SEQ ID NO: 32 | | | | | change |
| 14. | VLGAEEAATSYW | 1, 2, 4, 7, 8 | 5, 6 | −151.8 | −34.7 | Instable |
| | SEQ ID NO: 33 | | | | | |
| 15. | VLGREEWAAAYW | 1, 2, 8, 9, 10 | 4,5, 6 | −192.2 | 5.7 | Stable |
| | SEQ ID NO: 34 | | | | | |
| 16. | VLAREEAAAAAA | 1, 2, 3, 7, 8, | 4, 5, 6 | −136.2 | −50.3 | Instable |
| | SEQ ID NO: 35 | 9, 10, 11, 12 | | | | |
| 17. | VLAREEASAAAA | 1, 2, 3, 7, 8, | 4, 5, 6 | −136.2 | −50.3 | Instable |
| | SEQ ID NO: 36 | 9, 10, 11, 12 | | | | |
| 18. | AAGAAAWSTSYW | 1, 2, 4, 5, 6 | — | −190.2 | 3.7 | No |
| | SEQ ID NO: 37 | | | | | change |
| 19. | AAGAAAWAASYW | 1, 2, 4, 5, 6, | 1 | −185.9 | 0.6 | No |
| | SEQ ID NO: 38 | 8, 9 | | | | change |

TABLE 14
Alanine mutant Docking study analysis.

| S. | | N-terminus | | | | | | C-terminus | | | | | |
|---|---|---|---|---|---|---|---|---|---|---|---|---|---|
| No | MUTANTS | 1 | 2 | 3 | 4 | 5 | 6 | 7 | 8 | 9 | 10 | 11 | 12 |
| 1 | Alanine | X1 | X2 | G | X4 | X5 | X6 | W | X8 | T | S | Y | W |
| | mutant | | | | | | | | | | | | |
| | docking study | | | | | | | | | | | | |
| 2 | Triple mutant | V | X2 | X3 | R | X5 | X6 | X7 | X8 | T | X10 | Y | W |
| | study | | | | | | | | | | | | |
| | (3, 7, 8 vs | | | | | | | | | | | | |
| | 4, 7, 8) | | | | | | | | | | | | |
| 3 | Multi mutant | V | L | X3 | R | E | E | X7 | X8 | X9 | X10 | X11 | X12 |
| | study | | | | | | | | | | | | |
| | Towards C-ter | | | | | | | | | | | | |
| | (3, 7, 8-12) | | | | | | | | | | | | |
| 4 | Multi mutant | V | L | X3 | R | X5 | X6 | X7 | S | T | X10 | X11 | W |
| | study | | | | | | | | | | | | |
| | Towards C-ter | | | | | | | | | | | | |
| | (3, 7, 9-12) | | | | | | | | | | | | |
| 5 | Hydrophobic | X1 | X2 | X3 | R | X5 | X6 | W | X8 | T | X10 | X11 | W |
| | Moments OF | | | | | | | | | | | | |
| | VLG-peptide | | | | | | | | | | | | |
| | best poses | | | | | | | | | | | | |
| 6 | Hydrophobic | V | L | X3 | R | E | E | X7 | X8 | X9 | X10 | Y | W |
| | interactions | | | | | | | | | | | | |
| | OF VLG- | | | | | | | | | | | | |
| | peptide best | | | | | | | | | | | | |
| | poses | | | | | | | | | | | | |
| 7 | Hydrogen | V | X2 | X3 | R | X5 | X6 | X7 | S | X9 | S | Y | W |
| | bond | | | | | | | | | | | | |
| | interactions | | | | | | | | | | | | |
| | OF VLG- | | | | | | | | | | | | |
| | peptide best | | | | | | | | | | | | |
| | poses | | | | | | | | | | | | |
| Result | Occurrence | 5/7 | 3/7 | 1/7 | 6/7 | 2/7 | 2/7 | 2/7 | 2/7 | 4/7 | 2/7 | 4/7 | 6/7 |

In conclusion, it can be considered that the positions, 1 (V), 4 (R), 9 (T), 11 and 12 (YW) are important for the binding according to the alanine mutant docking analysis.

TABLE 15

| | |
|---|---|
| Stage 4: | VLGREEWSTSYW |
| Alanine mutant | (SEQ ID NO: 4) |
| docking study | |

### 2.2.2. Hydrophobic Moments

The hydrophobic moment of the alanine mutated peptides will generally point to the interior of the protein. So the side chains of hydrophobic residues are oriented towards the interior of a protein. Besides, the hydrophobic moment of a residue is proportional to the hydrophobicity of the residue and whose direction is dependent on the side chain orientation. The hydrophobic moments of side chains of residues forming a secondary structure element can give indication of the preferred orientation of the region of protein.

Except for 2, 5, 6, Aug. 9, 2010 positions mutated to Ala, even though the flip in the peptide conformation led to stable protein-peptide complex which is much similar/slightly better ΔΔG value of −5.7 to −12.6 kcal/mol, it may account for an analogous stability value possibly due to 1-3 hydrogen bonds (˜−4.5 kcal/mol per H-bond). This is due to conversion of amphipathic, and hydrophilic residues to hydrophobic in nature. Yet the C and N terminal residues will proceed by adopting hydrophobic nature while binding with the active site of the EGFRvIII protein.

TABLE 16
Peptide residue specificity of all mutants with EGFRvIII protein.

| Peptide | | | | | | | | |
|---|---|---|---|---|---|---|---|---|
| residues | | EGFRvIII residues | | | | | | |
| VAL1 | NP | GLU400 | GLU397 | CYS287 | GLU530 | ARG300 | ARG509 | GLU376 |
| LEU2 | NP | GLY508 | GLU397 | SER529 | GLU293 | LYS375 | ARG310 | |
| GLY3 | NP | ARG300 | GLU376 | GLU293 | LYS375 | SER340 | | |
| ARG4 | P+ | GLU400 | SER506 | GLU530 | THR339 | SER529 | GLU376 | GLU295 |
| ALA4 | NP | LYS375 | | | | | | |
| GLU5 | P− | ASN528 | MET294 | GLU295 | GLU293 | LYS375 | ARG310 | |
| GLU6 | P− | ARG310 | ASN398 | ARG427 | ARG497 | LYS455 | LYS375 | ARG509 |
| TRP7 | NP | LYS430 | ARG427 | | | | | |
| SER8 | P | GLU400 | LYS375 | | | | | |
| THR9 | P | GLU400 | | | | | | |
| ALA9 | NP | ARG509 | ARG310 | | | | | |
| SER10 | P | GLU376 | ARG300 | ARG310 | | | | |
| ALA10 | NP | ASN398 | | | | | | |
| TYR11 | P | THR339 | ARG310 | GLU376 | GLU293 | SER340 | PRO308 | |
| TRP12 | NP | GLU397 | ARG300 | ARG509 | GLU376 | TYR292 | ASN398 | ARG427 |

| Peptide | | | | | | |
|---|---|---|---|---|---|---|
| residues | | EGFRvIII residues | | | | |
| VAL1 | NP | ASN398 | SER342 | GLU293 | ARG503 | ARG310 |
| LEU2 | NP | | | | | |
| GLY3 | NP | | | | | |
| ARG4 | P+ | ASN504 | ARG310 | | | |
| ALA4 | NP | | | | | |
| GLU5 | P− | | | | | |
| GLU6 | P− | | | | | |
| TRP7 | NP | | | | | |
| SER8 | P | | | | | |
| THR9 | P | | | | | |
| ALA9 | NP | | | | | |
| SER10 | P | | | | | |
| ALA10 | NP | | | | | |
| TYR11 | P | | | | | |
| TRP12 | NP | ARG497 | GLU293 | SER340 | SER291 | ARG310 |

The Table 16 gives the overall look about which peptide residues show affinity to which protein residues. The dark grey color boxes represent hydrophobic residue; light grey boxes represent hydrophilic residues.

Overall, the alanine scan resulted peptides in N-ter to C-ter, flipped the conformation of the peptide while using C-terminal polar residues to bind with the large cavity of the protein, while the VLGREEWSTSYW (SEQ ID NO: 4) peptide uses the hydrophobic N-terminal region to bind with the large binding cavity of the protein.

All the 19 mutated peptides shown varying affinities with the EGFRvIII protein. The peptide residues that have shown binding with the receptor 1 (V), 4 (R), 6 (E) and 12 (W) are found to be crucial for the complex formation.

TABLE 17

| | | |
|---|---|---|
| Stage 5: | VLGREEWSTSYW | With respect |
| Specific | (SEQ ID NO: 4) | to all 19 Ala- |
| Residues | | mutated peptides |
| to bind | | |
| protein (with | | |
| respect to | | |
| mutants) | | |

### 2.3. Conclusion

Based on the observations, the below given residues are highly important to bind with EGFRvIII protein even on mutated state. In the present study, molecular docking, mutational redocking, molecular dynamics and affinity maturation studies, conducted with the VLGREEWSTSYW (SEQ ID NO: 4) peptide, concludes to the importance of the residues 1 (V), 4 (R), 6 (E), 7 (W), 11 and 12 (YW) of VLGREEWSTSYW (SEQ ID NO: 4) peptide for making a stable complex with EGFRvIII protein.

TABLE 18
summary of the consensus sequence prediction.

| | | |
|---|---|---|
| Stage 1: Docking | VLGREEWSTSYW | Local |
| | (SEQ ID NO: 4) | confidence/ |
| Stage 2: Affinity maturation | VLGREEWSTSYW | best |
| | (SEQ ID NO: 4) | poses |
| Stage 3: Molecular Dynamics | VLGREEWSTSYW | |
| | (SEQ ID NO: 4) | |
| Stage 4: Alanine mutant docking | VLGREEWSTSYW | confidence/best |
| study | (SEQ ID NO: 4) | poses |
| Stage 5: Specific Residues to bind | VLGREEWSTSYW | With respect to |
| protein (with respect to mutants) | (SEQ ID NO: 4) | all 19 Ala- |
| | | mutated peptides |
| Consolidated residue confidence | VLGREEWSTSYW | Overall Best |
| | (SEQ ID NO: 4) | residues |
| Best consensus sequence | V.X1.X2.R.X3.EW.X4.X5.X6.YW | |
| | (SEQ ID NO: 1) | |

### REFERENCES

Archer G E, Sampson J H, Lorimer I A, Mclendon R E, Kuan C T, Friedman A H, et al. Regional treatment of epidermal growth factor receptor vIII-expressing neoplastic meningitis with a single-chain immunotoxin, MR-1. Clin Cancer Res Off J Am Assoc Cancer Res. sept 1999; 5(9):2646-52.
Batra S K, Castelino-Prabhu S, Wikstrand C J, Zhu X, Humphrey P A, Friedman H S, et al. Epidermal growth factor ligand-independent, unregulated, cell-transforming potential of a naturally occurring human mutant EGFRvIII gene. Cell Growth Differ Mol Biol J Am Assoc Cancer Res. October 1995; 6(10):1251-9.
Boohaker R J, Lee M W, Vishnubhotla P, Perez J M, Khaled A R. The use of therapeutic peptides to target and to kill cancer cells. Curr Med Chem. 2012; 19(22):3794-804.
Cheng, L. et al. GE11-modified liposomes for non-small cell lung cancer targeting: preparation, ex vitro and in vivo evaluation. Int J Nanomedicine 9, 921-935(2014).
Cloughesy T F, Cavenee W K, Mischel P S. Glioblastoma: from molecular pathology to targeted treatment. Annu Rev Pathol. 2014; 9:1-25.
Dehn, S., Chapman, R., Jolliffe, K. A. & Perrier, S. Synthetic Strategies for the Design of Peptide/Polymer Conjugates. Polymer Reviews 51, 214-234 (2011).
Dejesus, O. T. Synthesis of [⁶⁴Cu] Cu-NOTA-Bn-GE11 for PET Imaging of EGFR-Rich Tumors. Curr. Radiopharm. 2012, 5 (1), 15-18.
Denholt C L, Binderup T, Stockhausen M-T, Poulsen H S, Spang-Thomsen M, Hansen P R, et al. Evaluation of 4-[¹⁸F]fluorobenzoyl-FALGEA-NH2 as a positron emission tomography tracer for epidermal growth factor receptor mutation variant Ill imaging in cancer. Nucl Med Biol. mai 2011; 38(4):509-15.
Denholt C L, Hansen P R, Pedersen N, Poulsen H S, Gillings N, Kjaer A. Identification of novel peptide ligands for the cancer-specific receptor mutation EFGRvIII using a mixture-based synthetic combinatorial library. Biopolymers. mars 2009; 91(3):201-6.
Dhankhar R, Vyas S P, Jain A K, Jain A K, Arora S, Rath G, et al. Advances in novel drug delivery strategies for breast cancer therapy. Artif Cells Blood Substit Immobil Biotechnol. 1 Oct. 2010; 38(5):230-49.
Ge H, Gong X, Tang C K. Evidence of high incidence of EGFRvIII expression and coexpression with EGFR in human invasive breast cancer by laser capture microdissection and
immunohistochemical analysis. Int J Cancer. 20 mars 2002; 98(3):357-61.
Guex N, Peitsch M C. SWISS-MODEL and the Swiss-PdbViewer: an environment for comparative protein modeling. Electrophoresis. déc 1997; 18(15):2714-23.
Gunsteren W F van. Biomolecular simulation: the GROMOS96 manual and user guide. Zürich; Groningen; Zürich: Biomos; Vdf, Hochschulverlag A G an der ETH Zürich; 1996.
Guo G, Gong K, Wohlfeld B, Hatanpaa K J, Zhao D, Habib A A. Ligand-Independent EGFR Signaling. Cancer Res. 1 sept 2015; 75(17):3436-41.
Hersh, J. et al. Peptide-Modified Biopolymers for Biomedical Applications. ACS Appl. Bio Mater. 4, 229-251 (2021).
Hossein-Nejad-Ariani H, Althagafi E, Kaur K. Small Peptide Ligands for Targeting EGFR in Triple Negative Breast Cancer Cells. Sci Rep. December 2019; 9(1):2723.
Huang H-J, Nagane M, Klingbeil C, Lin H, Nishikawa R, Ji X-D, et al. Huang H S, Nagane M, Klingbeil C K, Lin H, Nishikawa R, Ji X D, Huang C M, Gill G N, Wiley H S, Cavenee WKThe enhanced tumorigenic activity of a mutant epidermal growth factor receptor common in human cancers is mediated by threshold levels of constitutive tyrosine phosphorylation and unattenuated signaling. J Biol Chem 272:2927-2935. J Biol Chem. 1 févr 1997; 272:2927-35.
Ian W. Hamley; Biomacromolecules 2014 15(5), 1543-1559. DOI: 10.1021/bm500246w.
Jiao, H.; Zhao, X.; Han, J.; Zhang, J.; Wang, J. Synthesis of a Novel ⁹⁹mTc Labeled GE11 Peptide for EGFR SPECT Imaging. Int. J. Radiat. Biol. 2020, 96 (11), 1443-1451.
Kuan C T, Wikstrand C J, Bigner D D. EGF mutant receptor vIII as a molecular target in cancer therapy. Endocr Relat Cancer. juin 2001; 8(2):83-96.
Li, X.; Hu, K.; Liu, W.; Wei, Y.; Sha, R.; Long, Y.; Han, Y.; Sun, P.; Wu, H.; Li, G.; Tang, G.; Huang, S. Synthesis and Evaluation of [¹⁸F]FP-Lys-GE11 as a New Radiolabeled Peptide Probe for Epidermal Growth Factor Receptor (EGFR) Imaging. Nucl. Med. Biol. 2020, 90-91, 84-92.
Li, X., Jian, M., Sun, Y., Zhu, Q. & Wang, Z. The Peptide Functionalized Inorganic Nanoparticles for Cancer-Related Bioanalytical and Biomedical Applications. Molecules 26, 3228 (2021)
Lin N U. Targeted therapies in brain metastases. Curr Treat Options Neurol. janv 2014; 16(1):276.
Lorimer I a. J. Mutant epidermal growth factor receptors as targets for cancer therapy. Curr Cancer Drug Targets. juin 2002; 2(2):91-102.
Lu C, Mi L-Z, Grey M J, Zhu J, Graef E, Yokoyama S, et al. Structural evidence for loose linkage between ligand binding and kinase activation in the epidermal growth factor receptor. Mol Cell Biol. November 2010; 30(22):5432-43.
Mao J, Ran D, Xie C, Shen Q, Wang S, Lu W. EGFR/EGFRvIII Dual-Targeting Peptide-Mediated Drug Delivery for Enhanced Glioma Therapy. ACS Appl Mater Interfaces. 26 juill 2017; 9(29):24462-75.
Mero, Anna, and Monica Campisi. 2014. “Hyaluronic Acid Bioconjugates for the Delivery of Bioactive Molecules” Polymers 6, no. 2:346-369.
Modjtahedi H, Moscatello D K, Box G, Green M, Shotton C, Lamb D J, et al. Targeting of cells expressing wild-type EGFR and type-III mutant EGFR (EGFRvIII) by anti-EGFR MAb ICR62: a two-pronged attack for tumour therapy. Int J Cancer. 10 juin 2003; 105(2):273-80.
Moscatello D K, Montgomery R B, Sundareshan P, McDanel H, Wong M Y, Wong A J. Transformational and altered signal transduction by a naturally occurring mutant EGF receptor. Oncogene. 4 juill 1996; 13(1):85-96.
Omidfar K, Rasaee M J, Modjtahedi H, Forouzandeh M, Taghikhani M, Golmakani N. Production of a Novel Camel Single-Domain Antibody Specificfor the Type III Mutant EGFR. Tumor Biol. 2004; 25(5-6):296-305.
Pedersen M W, Meltorn M, Damstrup L, Poulsen H S. The type III epidermal growth factor receptor mutation. Biological significance and potential target for anti-cancer therapy. Ann Oncol Off J Eur Soc Med Oncol. juin 2001; 12(6):745-60.
Pettersen E F, Goddard T D, Huang C C, Couch G S, Greenblatt D M, Meng E C, et al. UCSF Chimera—A visualization system for exploratory research and analysis. J Comput Chem. 2004; 25(13):1605-12.
Rahmanian, N.; Hosseinimehr, S. J.; Khalaj, A.; Noaparast, Z.; Abedi, S. M.; Sabzevari, O. ⁹⁹mTc-Radiolabeled GE11-Modified Peptide for Ovarian Tumor Targeting. DARU J. Pharm. Sci. 2017, 25, 13. https://doi.org/10.1186/s40199-017-0179-8.
Riaz, M. K. et al. Surface Functionalization and Targeting Strategies of Liposomes in Solid Tumor Therapy: A Review. Int J Mol Sci 19, 195(2018).
Striese, F.; Sihver, W.; Gao, F.; Bergmann, R.; Walther, M.; Pietzsch, J.; Steinbach, J.; Pietzsch, H.-J. Exploring Pitfalls of ⁶⁴Cu-Labeled EGFR-Targeting Peptide GE11 as a Potential PET Tracer. Amino Acids 2018, 50 (10), 1415-1431.
Tian W, Chen C, Lei X, Zhao J, Liang J. CASTp 3.0: computed atlas of surface topography of proteins. Nucleic Acids Res. 2 juill 2018; 46(W1): W363-7.
Xu, W.-W., Liu, D., Cao, Y. & Wang, X. GE11 peptide-conjugated nanoliposomes to enhance the combinational therapeutic efficacy of docetaxel and siRNA in laryngeal cancers. Int J Nanomedicine 12, 6461-6470 (2017).
Yang W, Wu G, Barth R F, Swindall M R, Bandyopadhyaya A K, Tjarks W, et al. Molecular targeting and treatment of composite EGFR and EGFRvIII-positive gliomas using boronated monoclonal antibodies. Clin Cancer Res Off J Am Assoc Cancer Res. 1 févr 2008; 14(3):883-91.
Zhou Y, Li C, Peng J, Xie L, Meng L, Li Q, et al. DNA-Encoded Dynamic Chemical Library and Its Applications in Ligand Discovery. J Am Chem Soc [Internet]. 1 Nov. 2018 [cité 12 Oct. 2021]; Disponible sur: https://pubs.acs.org/doi/pdf/10.1021/jacs.8b09277

SEQUENCE LISTING

| SEQ ID NO | Type | Sequence |
|---|---|---|
| SEQ ID NO: 1 | prot | V-X1-X2-R-X3-E-W-X4-X5-X6-Y-W |
| | | wherein each of X1, X2, X3, X4, |
| | | X5, and X6 independently |
| | | corresponds to any amino acid. |
| SEQ ID NO: 2 | prot | V-X1-X2-R-X3-E-W-S-X5-X6-Y-W |
| | | wherein each of X1, X2, X3, X5, |
| | | and X6 independently |
| | | corresponds to any amino acid. |
| SEQ ID NO: 3 | prot | V-X1-X2-R-X3-E-W-S-T-X6-Y-W |
| | | wherein each of X1, X2, X3, |
| | | and X6 independently |
| | | corresponds to any amino acid. |
| SEQ ID NO: 4 | prot | VLGREEWSTSYW |
| SEQ ID NO: 5 | prot | FALGEA |
| SEQ ID NO: 6 | prot | YHWYGYTPENVI |
| SEQ ID NO: 7 | DNA | ATGCAAGGTACCTTTCTATTCTCACTCT |
| SEQ ID NO: 8 | DNA | GTATGGGATTTTGCTAAACAAC |
| SEQ ID NO: 9 | DNA | CTCTTCCGATCTATGCCTTACGGATCCTCATTAATGCAAGCAATT |
| | | CCTTTAGTGGTACCTTTCTATTCTCAC |
| SEQ ID NO: 10 | DNA | CTCTTCCGATCTATGCCTTACGGATCCTCATTAATAAGCGCAATT |
| | | CCTTTAGTGGTACCTTTCTATTCTCAC |
| SEQ ID NO: 11 | DNA | CTCTTCCGATCTATGCCTTACGGATCCTCATTAGCATAAGCAATT |
| | | CCTTTAGTGGTACCTTTCTATTCTCAC |
| SEQ ID NO: 12 | DNA | CTCTTCCGATCTATGCCTTACGGATCCTCATTATATAGCGCAATT |
| | | CCTTTAGTGGTACCTTTCTATTCTCAC |
| SEQ ID NO: 13 | DNA | GTTTCGGCCGAACCACCACC |
| SEQ ID NO: 14 | DNA | AATGATACGGCGACCACCGAGATCTACACGCTTGTCAACACTCTT |
| | | TCCCTACACGACGCTCTTCCGATCTATGCCTTACGGATCCTCATT |
| | | A |
| SEQ ID NO: 15 | DNA | CAAGCAGAAGACGGCATACGAGATATCACGACGTGACTGGAGTT |
| | | CAGACGTGTGCTCTTCCGATCTGTTTCGGCCGAACCACCACC |
| SEQ ID NO: 16 | prot | LEKGNTLSTSTV |
| SEQ ID NO: 17 | prot | NPIVRSAEDGQL |
| SEQ ID NO: 18 | prot | NESGITRIALQD |
| SEQ ID NO: 19 | prot | YDADMFYMKTNM |
| SEQ ID NO: 20 | prot | ALGREEWSTSYW |
| SEQ ID NO: 21 | prot | VAGREEWSTSYW |
| SEQ ID NO: 22 | prot | VLAREEWSTSYW |
| SEQ ID NO: 23 | prot | VLGAEEWSTSYW |
| SEQ ID NO: 24 | prot | VLGRAEWSTSYW |
| SEQ ID NO: 25 | prot | VLGREAWSTSYW |
| SEQ ID NO: 26 | prot | VLGREEASTSYW |
| SEQ ID NO: 27 | prot | VLGREEWATSYW |
| SEQ ID NO: 28 | prot | VLGREEWSASYW |
| SEQ ID NO: 29 | prot | VLGREEWSTAYW |
| SSEQ ID NO: 30 | prot | VLGREEWSTSAW |
| SEQ ID NO: 31 | prot | VLGREEWSTSYA |
| SEQ ID NO: 32 | prot | VLAREEAATSYW |
| SEQ ID NO: 33 | prot | VLGAEEAATSYW |
| SEQ ID NO: 34 | prot | VLGREEWAAAYW |
| SEQ ID NO: 35 | prot | VLAREEAAAAAA |
| SEQ ID NO: 36 | prot | VLAREEASAAAA |
| SEQ ID NO: 37 | prot | AAGAAAWSTSYW |
| SEQ ID NO: 38 | prot | AAGAAAWAASYW |
| SEQ ID NO: 39 | prot | GGGGG |
| SEQ ID NO: 40 | prot | CGGGG |
| SEQ ID NO: 41 | prot | CGGGS |
| SEQ ID NO: 42 | prot | VCQGT |
| SEQ ID NO: 43 | prot | CVKKCPR |
| SEQ ID NO: 44 | prot | LEEKKG |

1. A peptide of 12 to 30 amino acids, said peptide comprising the sequence SEQ ID NO: 1 represented by:
(SEQ ID NO: 1)
V-X1-X2-R-X3-E-W-X4-X5-X6-Y-W,
wherein each of X1, X2, X3, X4, X5, and X6 independently corresponds to any amino acid, and wherein said peptide binds to the EGF receptor variant III (EGFRvIII).

2. The peptide according to claim 1, wherein:
a) each of X1 and X2 is a hydrophobic amino acid; and/or
b) each of X3, X4, X5, and X6 is a hydrophilic amino acid.

3. The peptide according to claim 1, wherein said peptide has higher affinity for EGFRvIII than for wild type EGF receptor (EGFR WT).

4. The peptide according to claim 1, wherein X4 is selected from the group consisting of: T, S, N, Q, C, and U.

5. The peptide according to claim 1, wherein X5 is selected from the group consisting of: T, S, N, Q, C, and U.

6. The peptide according to claim 1, wherein:
a) X1 is selected from the group consisting of: V, A, I, M, G, P and L;
b) X2 is selected from the group consisting of: V, A, I, M, G, P and L;
c) X3 is selected from the group consisting of: E, D, Q, and N;
d) X6 is selected from the group consisting of: T, S, N, Q, C, and U; or
e) any combination of a) to d) above.

7. The peptide according to claim 1, wherein said peptide comprises the sequence SEQ ID NO: 4 represented by:
(SEQ ID NO: 4)
V-L-G-R-E-E-W-S-T-S-Y-W.

8. The peptide according to claim 1, wherein said peptide consists of 12 to 25 amino acids.

9. A nucleic acid encoding the peptide according to claim 1.

10. A vector comprising a nucleic acid according to claim 9.

11. A host cell comprising the nucleic acid according to claim 9.

12. A conjugate comprising the peptide according to claim 1 linked to another entity, optionally via a linker.

13. The conjugate according to claim 12, wherein when present said linker is:
a) a non-cleavable linker selected from the group consisting of: aliphatic chains, PEG, and poly(amino acid) derivatives; or
b) a cleavable linker selected from the group consisting of: chemical labile linkers, disulfide-containing reducible linkers, enzymatically cleavable linkers, and chemically and/or thermally cleavable.

14. The conjugate according to claim 12, wherein said other entity is selected from a radiolabel, a fluorophore, another peptide, a polymer, a copolymer, a nanoparticle, a drug, or any combination thereof.

15. The conjugate according to claim 12, wherein:
a) said radiolabel is selected from the group consisting of: ¹⁸F, ⁶⁴Cu, and ⁹⁹mTc;
b) said fluorophore is selected from the group consisting of: fluorescein isothiocyanate (FITC), phycoerythrin (PE), Allophycocyanin (APC), fluorophores from the cyanine family, fluorophores from AlexaFluor family, fluorophores from Atto family, and fluorophores from Dy family;
c) said other peptide is 3-8 amino acids long, or is a short peptide tags;
d) said polymer is selected from the group consisting of: polyethylene glycol (PEG), poly(amino acid)s and polysaccharides;
e) said copolymer is selected from the group consisting of: copolymers of PEG and another polymer;
f) said nanoparticle is selected form the group consisting of:
polymeric nanoparticles, including polymeric micelles, polymersomes, and polyplexes; and
lipid-based nanoparticles with conjugation on various lipids; and
inorganic nanoparticles; or
g) said drug is an anti-tumoral drug.

16. A method for:
a) detecting or quantifying EGFRvIII-expressing cells or EGFRvIII-expressing secreted extracellular vesicles (SEV) in a biological sample in vitro or ex vivo;
b) detecting or quantifying EGFRvIII-expressing cells or EGFRvIII-expressing SEVs in a subject in vivo;
b) sorting EGFRvIII-expressing cells or EGFRvIII-expressing SEVs from a biological sample in vitro;
c) in vitro targeting of EGFRvIII; or
d) in vitro delivery of an entity to an EGFRvIII-expressing cell,
comprising the use of the peptide according to claim 1 or of a conjugate comprising said peptide linked to another entity as an EGFRvIII-binding molecule.

17. A method for treating cancer in a subject in need thereof, comprising administering to said subject a therapeutically effective amount of the peptide according to claim 1 or a conjugate comprising said peptide linked to another entity, or a pharmaceutical composition comprising said peptide or conjugate and a pharmaceutically acceptable vehicle.

18. The peptide according to claim 4, wherein said peptide comprises the sequence of SEQ ID NO: 2 represented by:
(SEQ ID NO: 2)
V-X1-X2-R-X3-E-W-S-X5-X6-Y-W.

19. The peptide according to claim 5, wherein said peptide comprises the sequence of SEQ ID NO: 3 represented by:
(SEQ ID NO: 3)
V-X1-X2-R-X3-E-W-S-T-X6-Y-W.

20. The peptide according to claim 19, wherein:
a) X1 is selected from the group consisting of: V, A, I, M, G, P and L;
b) X2 is selected from the group consisting of: V, A, I, M, G, P and L;
c) X3 is selected from the group consisting of: E, D, Q, and N;
d) X6 is selected from the group consisting of: T, S, N, Q, C, and U; or
e) any combination of a) to d) above.

21. A host cell comprising the vector according to claim 10.


SEQUENCE LISTING (44 sequences)

SEQ ID NO: 1 - protein, 12 aa, synthetic construct
  REGION 1..12: note = general concensus sequence of EGFRvIII-binding peptide
  REGION 2..3: note = MISC_FEATURE - X is any amino acid
  SITE 5: note = MISC_FEATURE - X is any amino acid
  REGION 8..10: note = MISC_FEATURE - X is any amino acid
VXXRXEWXXXYW

SEQ ID NO: 2 - protein, 12 aa, synthetic construct
  REGION 1..12: note = intermediate consensus sequence of EGFRvIII-binding peptide
  REGION 2..3: note = MISC_FEATURE - X is any amino acid
  SITE 5: note = MISC_FEATURE - X is any amino acid
  REGION 9..10: note = MISC_FEATURE - X is any amino acid
VXXRXEWSXXYW

SEQ ID NO: 3 - protein, 12 aa, synthetic construct
  REGION 1..12: note = narrow consensus sequence of EGFRvIII-binding peptide
  REGION 2..3: note = MISC_FEATURE - X is any amino acid
  SITE 5: note = MISC_FEATURE - X is any amino acid
  SITE 10: note = MISC_FEATURE - X is any amino acid
VXXRXEWSTXYW

SEQ ID NO: 4 - protein, 12 aa, synthetic construct
  REGION 1..12: note = EGFRvIII-binding peptide
VLGREEWSTSYW

SEQ ID NO: 5 - protein, 6 aa, synthetic construct
  REGION 1..6: note = EGFR-WT and EGFRvIII-binding peptide
FALGEA

SEQ ID NO: 6 - protein, 12 aa, synthetic construct
  REGION 1..12: note = EGFR-WT-binding peptide
YHWYGYTPENVI

SEQ ID NO: 7 - DNA, 28 bp, synthetic construct
  misc_feature 1..28: note = qPCR forward primer for phage titration
  source 1..28: mol_type = other DNA
atgcaaggtacctttctattctcactct

SEQ ID NO: 8 - DNA, 22 bp, synthetic construct
  misc_feature 1..22: note = qPCR reverse primer for phage titration
  source 1..22: mol_type = other DNA
gtatgggattttgctaaacaac

SEQ ID NO: 9 - DNA, 72 bp, synthetic construct
  misc_feature 1..72: note = Primer NGS-1 used for PCR1 and PCR2 in NGS library preparation
  source 1..72: mol_type = other DNA
ctcttccgatctatgccttacggatcctcattaatgcaagcaattcctttagtggtacctttctattctcac

SEQ ID NO: 10 - DNA, 72 bp, synthetic construct
  misc_feature 1..72: note = Primer NGS-2 used for PCR1 and PCR2 in NGS library preparation
  source 1..72: mol_type = other DNA
ctcttccgatctatgccttacggatcctcattaataagcgcaattcctttagtggtacctttctattctcac

SEQ ID NO: 11 - DNA, 72 bp, synthetic construct
  misc_feature 1..72: note = Primer NGS-3 used for PCR1 and PCR2 in NGS library preparation
  source 1..72: mol_type = other DNA
ctcttccgatctatgccttacggatcctcattagcataagcaattcctttagtggtacctttctattctcac

SEQ ID NO: 12 - DNA, 72 bp, synthetic construct
  misc_feature 1..72: note = Primer NGS-4 used for PCR1 and PCR2 in NGS library preparation
  source 1..72: mol_type = other DNA
ctcttccgatctatgccttacggatcctcattatatagcgcaattcctttagtggtacctttctattctcac

SEQ ID NO: 13 - DNA, 20 bp, synthetic construct
  misc_feature 1..20: note = Primer NGS-rev used for PCR1 and PCR2 in NGS library preparation
  source 1..20: mol_type = other DNA
gtttcggccgaaccaccacc

SEQ ID NO: 14 - DNA, 91 bp, synthetic construct
  misc_feature 1..91: note = Primer seq fw used for PCR1 and PCR2 in NGS library preparation
  source 1..91: mol_type = other DNA
aatgatacggcgaccaccgagatctacacgcttgtcaacactctttccctacacgacgctcttccgatctatgccttacggatcctcatta

SEQ ID NO: 15 - DNA, 86 bp, synthetic construct
  misc_feature 1..86: note = Primer seq rev used for PCR1 and PCR2 in NGS library preparation
  source 1..86: mol_type = other DNA
caagcagaagacggcatacgagatatcacgacgtgactggagttcagacgtgtgctcttccgatctgtttcggccgaaccaccacc

SEQ ID NO: 16 - protein, 12 aa, synthetic construct
  REGION 1..12: note = Peptide selected from the NGS analysis, not showing specific binding to hEGFRvIII cells in flow cytometry
LEKGNTLSTSTV

SEQ ID NO: 17 - protein, 12 aa, synthetic construct
  REGION 1..12: note = Negative hit peptide
NPIVRSAEDGQL

SEQ ID NO: 18 - protein, 12 aa, synthetic construct
  REGION 1..12: note = Peptide selected from the NGS analysis, not showing specific binding to hEGFRvIII cells in flow cytometry
NESGITRIALQD

SEQ ID NO: 19 - protein, 12 aa, synthetic construct
  REGION 1..12: note = Peptide selected from the NGS analysis, not showing specific binding to hEGFRvIII cells in docking experiments
YDADMFYMKTNM

SEQ ID NO: 20 - protein, 12 aa, synthetic construct
  REGION 1..12: note = Mutant of EGFRvIII-binding peptide with alanine in position 1
ALGREEWSTSYW

SEQ ID NO: 21 - protein, 12 aa, synthetic construct
  REGION 1..12: note = Mutant of EGFRvIII-binding peptide with alanine in position 2
VAGREEWSTSYW

SEQ ID NO: 22 - protein, 12 aa, synthetic construct
  REGION 1..12: note = Mutant of EGFRvIII-binding peptide with alanine in position 3
VLAREEWSTSYW

SEQ ID NO: 23 - protein, 12 aa, synthetic construct
  REGION 1..12: note = Mutant of EGFRvIII-binding peptide with alanine in position 4
VLGAEEWSTSYW

SEQ ID NO: 24 - protein, 12 aa, synthetic construct
  REGION 1..12: note = Mutant of EGFRvIII-binding peptide with alanine in position 5
VLGRAEWSTSYW

SEQ ID NO: 25 - protein, 12 aa, synthetic construct
  REGION 1..12: note = Mutant of EGFRvIII-binding peptide with alanine in position 6
VLGREAWSTSYW

SEQ ID NO: 26 - protein, 12 aa, synthetic construct
  REGION 1..12: note = Mutant of EGFRvIII-binding peptide with alanine in position 7
VLGREEASTSYW

SEQ ID NO: 27 - protein, 12 aa, synthetic construct
  REGION 1..12: note = Mutant of EGFRvIII-binding peptide with alanine in position 8
VLGREEWATSYW

SEQ ID NO: 28 - protein, 12 aa, synthetic construct
  REGION 1..12: note = Mutant of EGFRvIII-binding peptide with alanine in position 9
VLGREEWSASYW

SEQ ID NO: 29 - protein, 12 aa, synthetic construct
  REGION 1..12: note = Mutant of EGFRvIII-binding peptide with alanine in position 10
VLGREEWSTAYW

SEQ ID NO: 30 - protein, 12 aa, synthetic construct
  REGION 1..12: note = Mutant of EGFRvIII-binding peptide with alanine in position 11
VLGREEWSTSAW

SEQ ID NO: 31 - protein, 12 aa, synthetic construct
  REGION 1..12: note = Mutant of EGFRvIII-binding peptide with alanine in position 12
VLGREEWSTSYA

SEQ ID NO: 32 - protein, 12 aa, synthetic construct
  REGION 1..12: note = Mutant of EGFRvIII-binding peptide with alanine in positions 3, 7 and 8
VLAREEAATSYW

SEQ ID NO: 33 - protein, 12 aa, synthetic construct
  REGION 1..12: note = Mutant of EGFRvIII-binding peptide with alanine in positions 4, 7 and 8
VLGAEEAATSYW

SEQ ID NO: 34 - protein, 12 aa, synthetic construct
  REGION 1..12: note = Mutant of EGFRvIII-binding peptide with alanine in positions 8 to 10
VLGREEWAAAYW

SEQ ID NO: 35 - protein, 12 aa, synthetic construct
  REGION 1..12: note = Mutant of EGFRvIII-binding peptide with alanine in positions 3 and 7 to 12
VLAREEAAAAAA

SEQ ID NO: 36 - protein, 12 aa, synthetic construct
  REGION 1..12: note = Mutant of EGFRvIII-binding peptide with alanine in positions 3 7, and 9 to 12
VLAREEASAAAA

SEQ ID NO: 37 - protein, 12 aa, synthetic construct
  REGION 1..12: note = Mutant of EGFRvIII-binding peptide with alanine in positions 1, 2 and 4 to 6
AAGAAAWSTSYW

SEQ ID NO: 38 - protein, 12 aa, synthetic construct
  REGION 1..12: note = Mutant of EGFRvIII-binding peptide with alanine in positions 1, 2, 4 to 6, and 8-9
AAGAAAWAASYW

SEQ ID NO: 39 - protein, 5 aa, synthetic construct
  REGION 1..5: note = flexible peptide linker
GGGGG

SEQ ID NO: 40 - protein, 5 aa, synthetic construct
  REGION 1..5: note = flexible peptide linker
CGGGG

SEQ ID NO: 41 - protein, 5 aa, synthetic construct
  REGION 1..5: note = flexible peptide linker
CGGGS

SEQ ID NO: 42 - protein, 5 aa, synthetic construct
  REGION 1..5: note = wild-type EGFR peptide
VCQGT

SEQ ID NO: 43 - protein, 7 aa, synthetic construct
  REGION 1..7: note = wild-type EGFR peptide
CVKKCPR

SEQ ID NO: 44 - protein, 6 aa, synthetic construct
  REGION 1..6: note = wild-type EGFR peptide
LEEKKG